(19) United States
(12) Patent Application Publication
(10) Pub. No.: US 2023/0321308 A1
(43) Pub. Date: Oct. 12, 2023

(54) IRRADIATION DEVICE FOR UV IRRADIATION

(71) Applicant: Virobuster International GmbH, Windhagen (DE)

(72) Inventor: Fehmi Yigit, Gronau (DE)

(73) Assignee: Virobuster International GmbH, Windhagen (DE)

(21) Appl. No.: 18/001,822

(22) PCT Filed: Jun. 18, 2021
(86) PCT No.: PCT/EP2021/066577

§ 371 Date: Dec. 14, 2022

(30) Foreign Application Priority Data
Jun. 19, 2020 (EP) 20181097.5

(51) Int. Cl. A61L 9/20; C02F 1/32

(52) U.S. Cl. CPC A61L 9/20; C02F 1/325; C02F 2201/3222; A61L 2209/15; A61L 2209/14

(57) ABSTRACT

The invention relates to an irradiation device for UV irradiation, in particular UV-C irradiation, of a medium flowing through the irradiation device, in particular water or a gaseous medium, preferably air, in particular for the inactivation of microorganisms present in the medium, such as bacteria, germs, mould and/or viruses, having a housing through which the medium is to flow and which has an inlet and an outlet, and at least one radiation source arranged in the interior of the housing for irradiating the medium flowing through the housing, wherein the housing is reflective on the inner side facing the radiation source with a reflectance for the UV radiation emitted by the radiation source of at least 0.6, wherein the radiation source is arranged in the housing in such a way that the radiation emitted by the radiation source is reflected on the inner side of the housing.

### CROSS-REFERENCE TO RELATED APPLICATIONS

The present application is the U.S. national stage application of international application PCT/EP2021/066577, filed Jun. 18, 2021, which international application was published on Dec. 23, 2021, as International Publication WO 2021/255234 A2. The international application claims priority to European Patent Application No. 20181097.5, filed Jun. 19, 2020. The international application and European application are hereby incorporated by reference herein in their entireties.

### FIELD

The invention relates to an irradiation device for UV irradiation, in particular UV-C irradiation, of a medium flowing through the irradiation device, in particular water, air and/or a gas mixture and/or a vapor mixture, in particular for inactivating and/or killing microorganisms present in the medium, such as bacteria, germs, mold and/or viruses. The irradiation device has an inlet and an outlet for the medium or the medium flow, the inlet and the outlet being provided on a housing through which the medium flows. A radiation source emitting UV radiation, in particular UV-C radiation, is arranged inside the housing for irradiating the medium flowing through the housing.

### BACKGROUND

In particular, the present invention relates to the technical field of so-called UV disinfection. The term “UV disinfection” refers to processes in which microorganisms—also known as microbes—can be killed by treatment with UV radiation. UV disinfection can be used for drinking water treatment, surface disinfection, exhaust air treatment, but also in the field of hygienic food processing or similar. Air in public areas can also be kept “clean” in this way, i.e. with a low level of harmful microorganisms.

In the following, UV-C radiation is understood to be radiation with a wavelength between 100 nm and 280 nm, in particular UV-C radiation at a wavelength between 200 nm and 280 nm, preferably 240 nm to 280 nm, being used in the context of UV disinfection.

UV disinfection uses in particular a wavelength of UV radiation between 200 and 300 nm. In this process, the emitted UV radiation has a bactericidal effect—that is, it is absorbed by DNA and destroys its structure, inactivating living cells. In this way, microorganisms such as viruses, bacteria, yeasts and fungi can be rendered harmless with UV radiation within a very short time, especially within a few seconds.

With sufficiently high irradiance, UV disinfection is thus a reliable and ecological method, since in particular no addition of further chemicals is necessary. It is particularly advantageous that microorganisms cannot develop resistance to UV radiation. Finally, UV disinfection can also interrupt the reproduction of microorganisms.

The UV disinfection process can also be referred to as “Ultraviolet Germicidal Irritation” (UVGI) and/or microbial disinfection, in particular using UV radiation with a wavelength of 254 nm. The use of UV disinfection is particularly advantageous for virus inactivation in air purification, as it enables large volumes of air to be freed from viruses.

The outbreak of the Corona pandemic in early 2020 demonstrates the urgent need to have economical, efficient and ecological virus inactivation processes in place.

The aim of further developments is to provide devices that enable media streams with a large throughput, especially air streams, to be cleaned efficiently. A further or alternative goal is to also kill those particles that are comparatively resistant to UV radiation. Ultimately, various basic radiation doses are known, which may differ depending on the microorganisms to be killed.

The UV radiation dose (J/m²) is obtained by multiplying the UV irradiance (W/m²) and the UV irradiation time (s).

Ultimately, the inactivation of microorganisms depends in particular on the power or the achieved intensity of the UV radiation. The distance of the medium flow to the radiation source also has an influence on the efficiency of the disinfection and/or cleaning.

### SUMMARY

The object of the present invention is to provide an improved irradiation device for UV irradiation, in particular UV-C irradiation, which preferably enables more efficient inactivation of microorganisms.

The aforementioned object is solved according to the invention by an irradiation device for UV irradiation, in particular UV-C irradiation, of a medium flowing through the irradiation device, the irradiation device having a housing which has an inlet and an outlet for the medium and through which the medium flows, and at least one radiation source which is arranged in the interior of the housing and emits UV radiation for irradiating the medium flowing through the housing. On the inner side facing the radiation source, the housing is designed to be reflective at least in areas, preferably over the entire surface, with a reflectance for the UV radiation emitted by the radiation source of at least 0.6.

According to the invention, the radiation source is arranged in the housing in such a way that the radiation emitted by the radiation source is, preferably directed, reflected on the inner side of the housing and that the radiation emitted by the radiation source constructively interferes with the, preferably directed, reflected radiation.

Alternatively or additionally, according to the invention, it is provided that the radiation source is arranged in the housing in such a way that the radiation emitted by the radiation source is, preferably directed, reflected on the inside of the housing and that the radiation emitted by the radiation source to the, preferably directed, reflected radiation has a path difference differing from an integer multiple of half the wavelength and/or of half the wavelength. A path difference of the in particular coherent waves and/or radiations of the, preferably directed, reflected radiation and the radiation emitted by the radiation source, which corresponds to an integer multiple of half the wavelength and/or of half the wavelength, would in particular cause destructive interference. Alternatively or additionally, it may also be provided that the radiation emitted by the radiation source interferes at least substantially non-destructively with the, preferably directed, reflected radiation, in particular wherein less than 30%, preferably less than 25% and in particular between 0% to 20%, of the radiation emitted by the radiation source interferes destructively.

According to the invention, the irradiation device can be used to inactivate and/or kill microorganisms present in the medium, such as bacteria, germs, mold and/or viruses. Air may be provided as the medium. Alternatively or additionally, water, gas or a vapor mixture may be provided as the medium.

In particular, the radiation source is arranged in the housing in such a way that the radiation, preferably emitted by the radiation source, is disturbed as little as possible. Preferably, a low interference and/or adverse influence on the radiation, preferably emitted by the radiation source, is achieved by preventing and/or reducing deconstructive interference and/or the amount of destructive interference.

In the present application, a distinction is made between directed reflection—also called direct reflection—and diffuse reflection. In particular, the inner side of the housing on which the radiation is reflected is designed in such a way that a non-diffuse, namely in particular a directed and/or direct, reflection of the incoming radiation takes place. For this, in particular, an at least substantially smooth surface of the inner side of the housing is required, which in particular is just not rough, which would otherwise cause a diffuse reflection.

Furthermore, according to the invention, the radiation source is arranged in particular in such a way that the constructive interference is used specifically to increase the efficiency of the irradiation device. In this context, different designs of the irradiation device according to the invention are conceivable, so that an interference pattern based on constructive interference can be achieved. In particular, either the housing wall, in particular an inner wall of the housing comprising the inner side, can be designed accordingly and/or the radiation source can be arranged in a certain manner relative to the inner wall of the housing.

In particular, the interior of the housing and/or the enclosed interior of the housing can be considered a UV treatment chamber for treating the medium.

Preferably, the inner wall is a reflector, which is further preferably formed as a housing component that is inserted into the housing and/or is replaceable.

For the purposes of the invention, the reflectance is understood as the ratio between reflected and incident intensity as a quantity of energy. The reflectance may depend in particular on the material of the inner wall on which the radiation impinges and on the radiation itself.

Interference describes the change in amplitude of two or more waves according to the superposition principle. Interference occurs in principle with different types of waves, wherein UV radiation is relevant for interference in the context of the invention.

Destructive interference denotes a phenomenon where at a certain place the waves cancel each other out. Constructive interference, on the other hand, characterizes those places where the waves—if they meet—amplify each other. This results in an increase of the amplitude.

According to the invention, the aim is in particular to increase the amplitude of the, in particular maximum, total intensity of the interacting UV radiation by constructive interference. Thus, an interference pattern characterized by constructive interference is created inside the housing. In those areas within the housing where constructive interference predominates, a particularly efficient killing of viruses can be made possible.

So far, in practice, the arrangement of the radiation source within the housing has been governed only by practical considerations. As has been found in experiments according to the invention, in arrangements of the radiation source within the housing known in practice, it is provided that the destructive interference exceeds the constructive interference.

Thus, it is known in the prior art in particular to arrange the longitudinal axis of the radiation source parallel and/or coaxial to the longitudinal axis of the housing. In most cases, the radiation source is arranged centrally in the housing.

The longitudinal axis of the housing or radiation source designates in particular that axis which runs in the direction of the longer or largest extension of the body and thus represents the axis running in the longitudinal direction. The longitudinal axis can—but does not have to—be an approximate axis of symmetry of the housing or radiation source.

According to the invention, it is also possible to kill bacteria, viruses and/or fungi with a comparatively high UV resistance.

Preferably, it is provided that the radiation source is arranged in the housing in such a way that in the interference pattern generated by interaction of the radiation emitted by the radiation source with the reflected radiation, the proportion of constructive interference exceeds the proportion of destructive interference, preferably by at least 10%, preferably by at least 20%, more preferably by at least 40%, more preferably by at least 50%, in particular by at least 70%.

Alternatively or additionally, it may be provided that the radiation source is arranged in the housing in such a way that the proportion of the interacting and constructively interfering radiation exceeds the proportion of the interacting and destructively interfering radiation, preferably by at least 10%, more preferably by at least 20%, more preferably by at least 40%, preferably by at least 50%, in particular by at least 70%.

Ultimately, an interference pattern is created in particular by the radiation emitted by the radiation source being reflected on the inner side of the housing and thus interacting with the emitted radiation of the radiation source and all the radiation located inside the housing—and/or in the UV treatment chamber. In the interference pattern achieved according to the invention, the advantages associated with constructive interference and/or low destructive interference can be specifically used to improve the efficiency of the entire device.

In a further preferred embodiment, it is provided that the radiation source is arranged in the housing in such a way that the interference pattern produced by interaction of the radiation emitted by the radiation source with the, preferably directed, reflected radiation has a, in particular averaged, maximum total intensity of the interference pattern, which is greater by at least 50%, preferably at least 100%, more preferably at least 200%, more preferably at least 280%, more preferably between 300% to 500%, than the maximum intensity of the direct radiation emitted by the radiation source before—in particular at least substantially directly—reflection at the inner side of the housing. In particular, the total intensity is achieved by superposition and/or constructive interference of the radiation interacting with each other in the housing, so constructively in particular the radiation emitted by the radiation source is superimposed on the, preferably directed, reflected radiation. Also, the reflected radiation can in particular be reflected several times, so that in particular a plurality of inter-reflections result, which can interact with each other in the interference pattern.

This increase in total intensity enables efficient killing of microorganisms, especially viruses, since the inactivation of microbes ultimately correlates in particular to the intensity of the radiation encountered by the microbes. Further influence is exerted by the time period during which the microbes are exposed to the UV radiation.

Thus, by increasing the intensity of UV radiation, the radiation dose can be increased, especially for the same duration of exposure of microorganisms to UV-C radiation.

In addition, the efficiency of UV disinfection is influenced by the resistance value of the microorganisms (k [m²/J]) to UV radiation, the exposure time (t [s]) and the intensity of the radiation (I [W/m²]). The resistance value k of the microorganisms cannot be influenced and can in particular be between 0.001 to 0.5 m²/J, preferably between 0.0014 to 0.3 m²/J.

According to the invention, it can be achieved that for a given UV resistance (k-factor), a low exposure time is sufficient by increasing the total intensity, since the variables ultimately influence each other in particular. Thus, both by increasing the exposure time and by increasing the intensity, the inactivation of the microorganisms after overcoming the UV resistance can be achieved.

In particular, the microorganism kill rate can be determined by the following formula:

S(t)=Sₒe^{-I·k·t}

with:
I=intensity (W/m²),
k=UV rate of the microorganism (m²/J),
t=the dwell time (s)
S=microbial content
Sₒ=initial microbial content.

In another very particularly preferred embodiment of the invention, it is provided that the inner side of the housing has a reflectance for the UV radiation emitted by the radiation source of at least 0.7, preferably of at least 0.8, further preferably of at least 0.9. In particular, the reflectance is selected such that the interference according to the invention can be caused, which can be caused by interaction of the radiation, preferably directed, reflected at the inner side of the housing with the radiation located inside the housing.

Particularly preferably, only a small proportion of the UV radiation is transmitted through the inner wall of the housing. Preferably, the transmitted portion of the UV radiation is less than 0.15, preferably less than 0.1, more preferably less than 0.05.

The inner wall of the housing and/or the inner side of the housing can be coated with a UV radiation-reflecting coating, which in particular ensures the desired degree of reflection of the inner side of the housing, at least in certain areas, preferably over the entire surface and/or completely. Alternatively or additionally, it can be provided that the inner wall and/or the inner side of the housing has metal as material and/or consists thereof. In particular, the inner wall may comprise and/or consist of a sheet metal, in particular a sheet metal comprising aluminum and/or a galvanized sheet metal and/or a stainless steel sheet metal. In this context, as previously explained, the inner wall of the housing can be formed as a component that can be inserted into the housing. Particularly preferably, a multiple-coated aluminum sheet is provided as the material for the inner wall of the housing.

Furthermore, in another preferred embodiment, it is provided that the inner wall and/or the inner side of the housing is cylindrical and/or corrugated or even—in particular flat and/or without elevations. Alternatively or additionally, it may be provided that the inner wall and/or the inner side of the housing has a plurality of elevations and/or depressions and/or that the inner wall of the housing is rotationally symmetrical. In further embodiments, the inner wall of the housing may also be non-rotationally symmetrical.

Ultimately, the inner wall and/or the inner side of the housing can be formed in such a way that a relative arrangement of the radiation source to the inner wall and/or inner side can be ensured, in which the highest possible proportion of constructive interference to the total interference image in the UV treatment chamber can be ensured.

In a further preferred embodiment, the longitudinal axis of the housing, in particular the longitudinal axis of the interior and/or treatment chamber of the housing and/or of the inner wall of the housing, is not aligned coaxially with the longitudinal axis of the radiation source. Particularly preferably, the radiation source is in particular not symmetrically arranged in the interior of the housing.

In another very particularly preferred embodiment, the longitudinal axis of the housing, in particular the longitudinal axis of the interior or treatment chamber of the housing and/or of the inner wall of the housing, is aligned offset, preferably obliquely, to the longitudinal axis of the radiation source. In particular, the longitudinal axis of the housing, in particular of the interior space of the housing and/or of the inner wall of the housing, is aligned with an angle α greater than 2°, preferably between 2° to 50°, more preferably between 3° to 15°, with respect to the longitudinal axis of the radiation source, and/or includes an angle α of the aforementioned order of magnitude with respect to the longitudinal axis of the radiation source.

Preferably, the radiation source is arranged decentrally—in particular non-centrally—in the and/or with respect to the housing. The decentralized arrangement can cause the constructive interference effects, since a positioning of the radiation source is made in particular in such a way that the, preferably directed, reflected radiation interferes constructively with the radiation emitted by the radiation source.

Furthermore, the at least one radiation source is preferably attached to the housing by means of a basic holding device in the interior of the housing. The holding device can in particular have a plurality of webs for fastening the end faces of the radiation source. Other arrangement possibilities of the radiation source within the housing are also conceivable. Ultimately, the basic holding device is preferably designed in such a way that the interference pattern formed by the interaction between the radiation emitted by the radiation source and/or the, in particular “old”, preferably directed, reflected radiation, preferably the previously, preferably directed, reflected radiation, and the radiation reflected at the inner wall of the housing, in particular now newly, preferably directed, reflected radiation is disturbed as little as possible.

Preferably, this low interference and/or low adverse influence is made possible by preventing and/or reducing deconstructive interference, in particular wherein the rays and/or the various portions of the radiation cannot at least substantially cancel each other out (destructive interference) and/or the portion of cancelled radiation and/or rays can be greatly reduced. The effect that the radiation is extinguished as little as possible is achieved in particular due to a lack of symmetry and/or a lack of symmetrical arrangement of the radiation source in the housing.

The decentralized and/or non-symmetrical and/or oblique arrangement of the radiation source within the housing can ensure that the wavelengths of the beams and/or of the different components of the radiation meeting each other cannot be offset by half a period and/or offset by a multiple of half the wavelength. Such an offset—which can also be referred to as a path difference—would otherwise cause destructive interference.

Alternatively or additionally, the basic holding means can also be designed to be reflective, at least in certain areas, and thus contribute in particular to increasing the constructive interference according to the invention. Particularly preferably, the basic holding device can hold the front ends of the radiation source with at least two, preferably at least three, basic holding means, which are designed in particular in a web-like manner and/or are fastened to the inner wall of the housing. Preferably, at least one end face of the radiation source is fixed to the inner wall of the housing by at least three web-like basic holding means. Thus, ultimately the radiation source can be supported within the housing. Preferably, the relative orientation of the radiation source to the inner wall of the housing can be predetermined by the basic holding means.

Preferably, the holding device is designed to adjust the orientation of the radiation source. The adjustability of the basic holding device can further make it possible to find in particular the position of the radiation source that allows the highest possible amount of constructive interference.

Preferably, the housing has a length between 30 to 200 cm, preferably between 80 to 150 cm. The housing may have a diameter, preferably an inner diameter, between 8 to 30 cm, preferably between 10 to 20 cm. In a particularly preferred embodiment, the housing has a length between 80 to 150 cm and an inner diameter between 10 to 20 cm. With the aforementioned dimensions, efficient inactivation of the microorganisms can be ensured in particular for a medium flow with a flow velocity between 1 to 2 m/s.

Furthermore, the irradiation device according to the invention can also be used in air conditioning systems or office buildings or the like. It is understood that the irradiation device can be designed depending on the medium flow and/or the flow rate of the medium flow.

Furthermore, in another preferred embodiment, it may be provided that the radiation source comprises a plurality of illuminants, preferably LEDs. Furthermore, alternatively or additionally, the radiation source may have an at least substantially elongated and/or rod-shaped form. In particular, the radiation source may have a length of at least 5 cm, preferably between 5 cm to 30 cm, more preferably between 10 cm to 20 cm. In a particularly preferred embodiment, it is provided that the radiation source has an elongated shape with a plurality of illuminants, preferably LEDs, arranged along an “array”.

In addition, the radiation source may have a diameter of at least 1 cm, preferably between 1 cm and 20 cm, more preferably between 2 cm and 10 cm, and in particular between 5 cm+/−1 cm. In particular, the diameter of the radiation source may also depend on the diameter of the illuminant used in the radiation source.

Alternatively or additionally, the radiation source can also be designed as a UV low-pressure lamp, in particular a low-pressure mercury discharge lamp, and/or as a UV medium-pressure lamp.

Finally, the radiation source can provide the UV radiation required to inactivate the microorganisms, in particular the UV-C radiation. In this context, the radiation source can have an intensity at its surface of between 1000 and 8000 W/m², preferably between 2000 and 6000 W/m² and in particular of 4200 W/m²+/−20%.

Furthermore, the radiation source can in particular provide a power of at least 100 W, preferably of 190 W+/−10%. The power in the UV-C radiation range may preferably be between 10 to 100 W, more preferably between 50 to 70 W. The radiation emitted by the radiation source can decrease with its intensity in the distance square. This reduction in amplitude can be counteracted by the constructive interference achieved.

In another preferred embodiment of the invention, it is provided that a plurality of radiation sources are arranged in the housing. In particular, between 2 to 10, preferably between 2 to 5, radiation sources may be arranged in the housing. The radiation sources can each in particular also have a plurality of illuminants, preferably LEDs. The plurality of radiation sources can provide radiation that is at least substantially uniform and/or sufficient to inactivate the microorganisms over the length of the housing.

Furthermore, the radiation from the multiple radiation sources can also interact with each other in the resulting interference pattern in the UV treatment chamber and contribute to increasing the constructive interference. The suitability for constructive interference according to the invention applies in particular to each radiation source used in the irradiation device according to the invention.

In principle, it is also possible for the radiation sources to be designed differently from one another. Preferably, the radiation sources are designed to be at least substantially identical.

Preferably, the plurality of radiation sources is arranged overlapping at least in some areas in the housing. The length of the overlapping area of at least two radiation sources may correspond to at least 20%, preferably at least 30%, more preferably between 30% to 80%, of the length of at least one radiation source. In particular, the overlap area may be selected as a function of the length of the housing and/or the width of the housing and/or as a function of the different radiation sources. Moreover, it has been found during the course of the invention that the overlapping range of the radiation sources in particular does not have a particularly detrimental effect on the constructive interference of the radiation according to the invention. Thus, ultimately, the power of the UV radiation within the housing can be increased by the multiple radiation sources. This also contributes to a more efficient and/or improved killing of the microbes.

Preferably, the multiple radiation sources, in particular at least two radiation sources, may be arranged at an angle and/or skew to each other. In an alternative or additional embodiment, it can also be provided that the longitudinal axes of the radiation sources are aligned at least substantially parallel to each other.

Particularly preferably, the longitudinal axes of the radiation sources are offset from each other, preferably at an angle. The longitudinal axes of the radiation sources can enclose an angle β between 1° and 90°, preferably between 2° and 50°, more preferably between 10° and 30°, with respect to each other. The angle β can be selected in particular as a function of the interference pattern caused within the housing, wherein the highest possible proportion of constructive interference can be achieved in the interference pattern.

In a further preferred embodiment, it is provided that the device comprises a fan for generating the medium flow from the inlet to the outlet. In particular, the fan is set in such a way that the medium flow can have a flow velocity of between 1 and 5 m/s, preferably between 1 and 2 m/s. In particular, the flow velocity can also depend on the internal diameter of the housing and/or on the interference figure achieved in the UV treatment chamber (that is, in the interior of the housing).

Preferably, the fan is located upstream of the UV treatment chamber and/or radiation source and/or the interior of the housing to create the flow of medium from the inlet to the outlet.

The medium flow can be guided at least essentially laminarly through the interior of the housing. Alternatively or additionally, however, it can also be provided that the medium flow in the housing and/or flowing through the housing forms a turbulent flow.

Furthermore, at least one radiation source can emit UV radiation in a wavelength range of at least 240 to 300 nm, preferably in a wavelength range of 250 to 285 nm, more preferably of 270 to 280 nm and in particular of 254 nm+/−10% and/or of 278 nm+/−10%. In the case of UV-C radiation with a wavelength of 254 nm+/−10%, a high level of virus inactivation can be achieved in particular. According to the invention, UV radiation with a wavelength in the aforementioned order of magnitude enables the microorganisms in the medium stream to be killed.

Preferably, a prefilter is arranged upstream of the inlet. The prefilter can preferably be designed in such a way that particles with a diameter greater than 1 μm, preferably greater than 0.5 μm, are filtered out of the medium flow. Thus, in particular, particles can be filtered out of the medium flow that would otherwise produce a so-called “shadow formation” in the UV treatment chamber during the resulting interaction with the UV radiation. Ultimately, it is relevant in this context that the UV radiation is in the order of magnitude between 0.2 to 0.3 μm. However, since the aforementioned particles, for example dust particles or pollen or the like, have a larger diameter than the wavelength, the wavelength in particular cannot pass through particles with a diameter greater than 1 μm. For example, a bacterium may have a diameter of approximately 0.3 μm. According to the invention, it has been found that the shadow effect is present in front of particles with a diameter smaller than 0.5 μm, in particular between 0.3 μm to 0.5 μm, but still the achieved killing of the microorganisms can be tolerated. Thus, those microorganisms with a diameter larger than the wavelength of the UV radiation can also be rendered harmless, since the UV radiation also hits them.

In the following, a further embodiment according to the invention is described, which in particular can be realized independently of the embodiment described above. In particular, it can be provided that previously described features, preferred embodiments, advantages and the like can also apply to the embodiment described below, although this is not explicitly described—to avoid unnecessary explanations.

The object according to the invention can also be solved by an irradiation device for UV irradiation, in particular UV-C irradiation, of a preferably gaseous and/or fluid medium, in particular water or air, flowing through the irradiation device, the irradiation device having a housing which has an inlet and an outlet for the medium and through which the medium is to flow. The housing can have the medium flowing through it. In particular, the irradiation device can be used to inactivate microorganisms present in the medium, such as bacteria, germs, mold and/or viruses. The irradiation device further comprises at least one radiation source arranged inside the housing and emitting UV radiation, in particular UV-C radiation, for irradiating the medium flowing through the housing.

The housing comprises a reflector, wherein the reflector on the inner side facing the radiation source is designed to be reflective at least in areas, preferably over the entire surface, with a reflectance for the UV radiation emitted by the radiation source of at least 0.6. In particular, the reflector can also be formed by the inner side of the housing. The inner side of the reflector and/or of the housing is in particular designed to be smooth in such a way that at least essentially a direct and/or directed reflection of the incoming radiation can take place.

The irradiation device has a holding device by means of which the at least one radiation source is held and/or fixed and/or can be held and/or fixed. The holding device is connected to the housing and/or the reflector, preferably detachably. The holding device can be designed in such a way that the central axis of the at least one radiation source encloses an angle to the central axis of the reflector.

According to the invention, the central axis is understood to be in particular the longitudinal axis of the reflector and/or the housing or the radiation source. The central axis lies and/or runs in particular in the respective center of the body and/or in the center of gravity of the respective body and preferably forms the axis of symmetry. Provided that the body is not symmetrical, the central axis of the respective body—that is of the reflector, of the housing and/or of the radiation source—forms the approximate axis of symmetry of the body. Thus, according to the invention, also central axes of such bodies are included which are not symmetrical.

In particular, the central axis of the radiation source or the reflector and/or the housing runs through the center of gravity and/or the center of the radiation source and/or the housing. The central axis preferably runs in longitudinal extension of the reflector and/or the housing or the radiation source, wherein the radiation source or the reflector and/or the housing is of elongated design.

A longitudinal extension is to be understood in particular in such a way that the length of the body exceeds the width of the body.

According to the invention, it has been found that by the aforementioned inclined arrangement between the central axis of the at least one radiation source and the central axis of the reflector and/or the housing, an increase in the constructive interference and thus, in particular, an improvement in the UV radiation dose to be administered with which the medium is treated can be achieved. The fact that such an improvement is achieved by an inclined position of the radiation source was not to be expected by the person skilled in the art.

Finally, according to the invention, it has been found that, in particular, the interference between the radiation directly reflected on the inner surface of the reflector and the radiation emitted by the radiation source can be controlled in a targeted manner, in particular in such a way as to result in an increase in the amplitude of the radiation intensity compared to a “straight” arrangement.

In addition, the radiation is directly reflected in particular several times on the inner side of the reflector, so that the oblique arrangement results—in an unpredictable way—in an increase of the intensity of the radiation, which leads to an improved UV dose. The formation of the resulting interference pattern is so complex that the resulting overall interference pattern cannot be reliably predicted and/or simulated without experimental tests, such as those carried out in accordance with the invention when the invention came into being—this is due in particular to the inter-reflections that occur in the reflector.

Finally, according to the invention, it is avoided that the radiation emitted by the radiation source is largely extinguished due to the reflection at the inner side of the reflector. The “extinction effects” in this respect are “accepted” in the prior art with the straight alignment of the radiation source present in the reflector, since ultimately it has not been known in the prior art how such extinction effects can be avoided. The fact that it is at all possible to increase the constructive interference has not been known to the skilled person in practice.

Preferably, the biological dose determined in particular on the basis of the view factor can be significantly increased, preferably by at least 5%, more preferably by at least 10%. Such an increase is achieved by the fact that, as explained before, the loss in the different reflection stages of the radiation emitted by the radiation source can be significantly lowered, namely due to the advantageous positioning of the radiation source in the reflector.

The oblique arrangement of the radiation source in the reflector thus makes it possible to increase the overall achieved killing result of the microorganisms present in the medium and thus to provide improved disinfection of the medium.

In a particularly preferred embodiment, it is provided that the included angle between the central axis of the at least one radiation source and the central axis of the reflector is between arcsin((0.2·D)/L) and arcsin((4·D)/L). Thus, in particular, the distance between one front end of the radiation source and the other front end, and/or the maximum displacement generated, can be between 0.2 D and 4 D. In this context, D indicates the, in particular maximum, diameter of the radiation source, where L references the length of the radiation source. In total, therefore, an oblique displacement of between 0.2·D and 4·D is achieved over the entire length of the respective radiation source.

In this context it is understood that the radiation sources (to each other) can have the same or also a different length or diameter. The aforementioned ratio of the angle refers to the respective length and the respective diameter of the considered radiation source. If the diameter of the radiation source varies, D denotes in particular the maximum and/or the average diameter.

Particularly preferably, the included angle between the central axis of the at least one radiation source and the central axis of the reflector is between arcsin((0.5·D)/L) and arcsin((3·D)/L), more preferably between arcsin(D/L) and arcsin((2·D)/L). Alternatively or additionally, it may be provided that the included angle between the central axis of the at least one radiation source and the central axis of the reflector is between 0.5° to 15°, more preferably between 2° to 10°, preferably between 2°+/−0.5°.

The aforementioned inclined position enables an improvement of the constructive interference and thus of the overall radiation pattern and/or interference pattern in the reflector. In the course of the invention, it has been surprisingly found that an increase in the maximum radiation intensity can be achieved by an oblique offset, which depends in particular on the diameter and length of the respective radiation source.

Furthermore, in another preferred embodiment, it is provided that the radiation source is at least substantially rod-shaped. Alternatively or additionally, it can be provided that the radiation source is at least substantially cylindrical and/or elongated. In particular, the longitudinal extension of the radiation source extends at least substantially—that is, taking into account the oblique and/or angular arrangement of the radiation source in the reflector—in the longitudinal direction of the housing and/or the reflector. Accordingly, the longitudinal extension of the radiation source runs in particular not orthogonally to the longitudinal extension of the housing and/or the reflector.

In addition, it may be provided that a plurality of radiation sources are held and/or fixed to the holding device. In particular, each central axis of each radiation source includes an angle, preferably of the order of magnitude mentioned above, to the central axis of the reflector.

In a further preferred embodiment, the central axes, in particular at least two central axes, more preferably at least four central axes, in particular all central axes, of the radiation sources are arranged parallel to each other. Alternatively or additionally, it can be provided that at least two central axes, preferably at least three central axes, more preferably at least four central axes, of the radiation sources are each arranged offset from one another, preferably obliquely. Accordingly, the radiation sources can also be arranged twisted, torted and/or warped with each other. In particular, the included angle between two adjacent radiation sources, in particular between the adjacent central axes of the adjacent radiation sources, can be between 1° to 120°, more preferably between 5° to 90°, more preferably between 10° to 40°. The aforementioned angle indicates in particular the degree of twisting between the radiation sources.

In the course of the invention, it has been surprisingly shown that a combination of the oblique position of the radiation source in the reflector and the additional torsion and/or twisting of the radiation sources with respect to each other enables such an interference pattern—generated by the radiation reflected at the reflector, preferably directionally, and the radiation emitted by the radiation source as well as the multiple reflections at the inner side of the reflector—which leads to a significant increase of the maximum intensity of the radiation located in the reflector. Ultimately, the oblique position of the radiation sources—both with respect to each other and with respect to the central axis of the reflector—leads to the fact that cancellation of the maxima (due to destructive interference) of the radiation emitted by the radiation source can be prevented and/or significantly reduced, so that the amplifying effects of the superposition of the radiation (namely, constructive interference) can be used. Exactly why such an interference effect and/or image results cannot be conclusively determined with certainty. The interference pattern that results depends on a variety of factors, and the radiation pattern that is produced also cannot be modeled with sufficient accuracy. Therefore, it has not been expected that there will be a significant improvement in the radiation dose to be administered and/or the maximum intensity achieved, in particular by at least 20%, compared to the prior art. However, according to the invention, such an improvement has been ensured by the oblique position of the radiation source(s).

Particularly preferably, the central axes of the radiation sources are arranged at an angle and/or skew to each other.

Furthermore, in another preferred embodiment, it is provided that the radiation source is detachably connected to the holding device. Preferably, each radiation source is detachably connected to the holding device. A detachable arrangement allows the advantage that a radiation source that is damaged or needs to be replaced can be removed from the holding device. In this regard, it may be provided that the radiation source is first removed from the reflector together with the holding device, with subsequent replacement of the radiation source.

In addition, preferably in a further embodiment of the idea of the invention, the holding device for at least one radiation source is provided with an adjusting means for adjusting the oblique position of the central axis of the radiation source with respect to the central axis of the reflector. The adjusting means can in particular be designed in such a way that it allows an adjustment in the released state and subsequently connects the radiation source firmly to the holding device by a fixing, so that in particular no adjustment of the radiation source can take place in the fixed state of the adjusting means. The adjusting means may comprise, for example, a screw connection. The adjusting means can also have a displacement device and/or the adjusting means is designed to be telescopic, at least in sections.

Furthermore, in another preferred embodiment, it is provided that the radiation sources are equally spaced from each other. In particular, the equal spacing of the radiation sources extends over the entire length of the respective radiation sources. Alternatively or additionally, it can be provided that, as explained before, at least two, in particular at least two directly adjacent, radiation sources enclose with their central axis a different angle to the central axis of the reflector. Accordingly, the radiation sources can also be arranged twisted and/or twisted with respect to each other, which, according to the invention, results in the previously mentioned advantageous effects of increasing the constructive interference.

Parallel spacing of the radiation sources is associated with the advantage of easy handling of the “lamp package”—i.e. the radiation sources attached to the holding device—as well as easy replacement of the individual radiation sources, since no attention has to be paid to possible twisting between the radiation sources.

Preferably, the holding device has a first holding unit. The first holding unit can be detachably connectable and/or connected to the housing and/or the reflector via a first connecting means of the holding device. Furthermore, the first holding device may comprise a plurality of first holding means spaced apart from each other at least in some areas. The holding means can in particular be designed as, preferably web-shaped, holding arms.

In this context, it is understood that the holding arms and/or the first holding means of the first holding unit can be designed differently from each other. The radiation sources can be fastened to the first holding means in such a way that in each case a first holding means is assigned to a radiation source and serves to fasten a radiation source.

A spacing of the radiation sources from each other can thus be achieved via an at least regionally provided spacing of the first holding means. In that the first holding means are assigned to the first holding unit and all first holding means are at least indirectly connected to one another, a compact design of the entire radiation unit—i.e. the radiation unit comprising the radiation sources—can be achieved. The radiation unit may also be referred to as a lamp package. The first holding unit therefore makes it possible to compactly remove the radiation unit from the housing and/or the reflector, ultimately enabling easy assembly and disassembly of the radiation unit—for example, for maintenance purposes—from the housing. The first holding unit also ensures that the radiation sources are always arranged in a fixed position in the housing and/or reflector with regard to their alignment.

By spacing the first holding means, in particular an at least substantially sun-shaped and/or star-shaped configuration of the first holding unit can be achieved, wherein the first holding means protrude starting from a common starting point.

Furthermore, the first holding means can be connected to a connection area of the first holding unit. The connection area can be connected to the connecting means, preferably directly, and/or be formed integrally therewith. Ultimately, the first holding means can be connected to the connecting means, and thus releasably connected to the housing and/or the reflector, via the connection area. The first holding means can be connected to the connection area by a respective end area. The further end area of the, preferably elongated, first holding means can be arranged freely—i.e. not be directly connected to a component of the first holding unit.

Ultimately, the first holding means can thus be designed as a supporting arm and/or cantilever arm, which is arranged and/or mounted at an end area on the connection area. The connection area can thus form the center of the first holding unit. However, the connection area does not have to be arranged in the center of gravity of the first holding unit, but can also be arranged outside the center of gravity of the first holding unit.

In addition, the radiation source can be connected to the first holding means, preferably at a front end area. The connection to the first holding means can, in particular, be designed to be detachable on the one hand and positive, non-positive and/or frictional on the other. In this context, it is understood that the end face of the radiation source does not necessarily have to be connected to the first holding means, but the front end area ultimately comprises the region of the radiation source that includes the end face. Thus, the radiation source is ultimately fixed to and/or connected to the first holding means in at least one end area.

The first holding means can also have at least one fastening means, for example at least one clip, at least one clamp, at least one spring leg or the like, for fastening with the radiation source, which is/are designed for releasable arrangement of the radiation source. This fastening means can furthermore preferably be arranged displaceably on the first holding means, in particular so that an adjustment of the arrangement of the radiation sources can take place. Alternatively or additionally, it can be provided that the fastening means is formed integrally with the further components of the first holding means. In this context, it is understood that the fastening means are considered to be a component of the first holding means.

In addition, according to the invention, the radiation source can be connected to the first holding means by different types of fastening.

Furthermore, it can be provided that the radiation source is accommodated in the holding means at least in certain areas, in particular so that the end face protrudes from the first holding means. The first holding means can also have corresponding fastening means, for example tension clamps, to hold the radiation source.

Moreover, in another preferred embodiment, it is provided that the first holding means, preferably all first holding means, is connected at one end area to the connection area. Alternatively or additionally, it can be provided that the first holding means is connected at its free end area—in particular the non-supported end area—to the radiation source. Also in this context, it is understood that the end area of the first holding means does not necessarily have to reference the outermost end of the first holding means, but the end area may comprise the outermost end and an adjoining region. In this case, the end area of the first holding means extends over at most 30%, preferably at most 20%, of the length of the elongated first holding means.

In a further preferred embodiment, it is provided that at least one first holding means, preferably at least two first holding means, preferably all first holding means, can be adjusted via a first adjusting means connected to the connection area. The first adjusting means can, for example, have a screw connection, preferably in combination with an elongated hole, and/or a telescopic adjustment facility or the like.

Finally, the first adjusting means can be designed in such a way that the oblique position of the central axis of the radiation source attached to the first holding means can be adjusted in relation to the central axis of the reflector. In this context, it is understood that the first adjusting means is “activated” and/or released only as needed. For example, in the released state, the first adjusting means can allow an adjustment of the inclined position of the central axis of the respective radiation source attached to the first holding means, wherein after the alignment has been completed, a renewed locking or securing of the first adjusting means can take place—for example, by a tightening of the screw connection. Thus, in the fixed state of the first adjusting means, the radiation source can be firmly arranged on the first holding means, in particular so that no renewed adjustment of the oblique position of the central axis of the radiation source is possible.

As explained above, the first holding means can be elongated. Alternatively or additionally, it may be provided that at least two first holding means have a length differing from each other. Alternatively or additionally, it can be provided that the first holding means has at least two arrangement areas, preferably for arranging the fastening means, for connection to the radiation source.

The arrangement areas can be used, for example, to achieve a staggered arrangement of the radiation sources in relation to one another. For example, a radiation source on the first holding means can be arranged further out (in relation to the free end area of the first holding means) than an immediately adjacent radiation source arranged on a further first holding means, but on an arrangement area offset in relation to the previously mentioned arrangement area.

Preferably, the first holding unit is designed in such a way that at least two angles enclosed between two directly adjacent first holding means are designed differently from each other and, in particular, deviate and/or differ from each other by at least 5%, preferably at least 10%. Particularly preferably, all first holding means are arranged relative to each other in such a way that all included angles between directly adjacent first holding means are different from each other. This can contribute to the further advantageous effect that constructive interference effects can be achieved by the oblique arrangement of the radiation sources in the reflector. This effect can now be further enhanced by the different arrangement of the first holding means, preferably at the first connection area.

Furthermore, the first holding unit can be designed to supply energy to the radiation sources. In particular, energy supply lines, in particular power lines, mains connection lines and the like, can be arranged in the first connecting means, in the first holding unit and/or in the first holding means. The power supply lines can be designed in such a way that the correct operation of the radiation sources, which are preferably designed as LEDs, can be ensured. The energy required for operating the radiation sources can thus be supplied to the radiation sources via the first holding means and/or via the first holding unit. Thus, the radiation sources arranged inside the reflector can be connected electrically and/or energetically.

In this context, it is understood that any ballasts, power supply connectors and the like provided can be arranged outside the reflector, in particular on the outside of the housing, and can be connected to the radiation sources via power supply lines which can be routed through the first holding unit. Accordingly, the advantageous routing of the lines can prevent power supply lines in the interior of the reflector, in particular unattached, from promoting possible destructive interference or from interfering with or even damaging the lamp package during assembly or disassembly of the radiation unit and/or the lamp package.

In a further preferred embodiment, it is provided that a second holding unit of the holding device is provided, preferably complementary and/or corresponding to the first holding unit. The second holding unit can be designed for holding or fixing the radiation sources. In particular, the radiation sources are at least indirectly connected to the second holding unit by means of the further front end area. The second holding unit can be connectable, preferably detachably, to the housing via at least one second connecting means of the holding device. Preferably, the second holding unit can have a plurality of second holding means which are spaced apart from one another at least in some areas, preferably designed as, in particular, web-shaped holding arms.

In this context, it is understood that the second holding arms and/or the second holding means of the second holding unit can be designed differently from each other. The radiation sources can be fastened to the second holding means in such a way that in each case a second holding means is assigned to a radiation source and serves to fasten a radiation source. Alternatively or additionally, it can be provided that a corresponding second holding means is assigned to each first holding means.

A spacing of the radiation sources from each other can thus be achieved via an at least regionally provided spacing of the second holding means. By the fact that the second holding means are assigned to the second holding unit and all second holding means are at least indirectly connected to each other, a compact design of the entire radiation unit—i.e. the radiation unit comprising the radiation sources—can be achieved. The second holding unit, together with the first holding unit, therefore enables the radiation unit to be compactly removed from the housing and/or the reflector and thus ultimately enables simple assembly and disassembly of the radiation unit—for example for maintenance purposes—from the housing.

By spacing the second holding means, in particular, an at least substantially sun-shaped and/or star-shaped configuration of the second holding unit can be achieved, wherein the second holding means protrude starting from a common starting point.

Furthermore, the second holding means may be connected to a second connection area of the second holding unit. The second connection area can be connected to the second connecting means, preferably directly, and/or be formed integrally therewith.

Ultimately, the second holding means can be connected to the second connecting means via the second connection area and thus detachably connected to the housing and/or the reflector. The second holding means can each be connected to the second connection area by one end area or be mounted thereon. The further end area of the, preferably elongated, second holding means can be arranged freely—that is, not be directly connected to a further component of the second holding unit.

Furthermore, the second holding means can thus be designed as a supporting arm and/or cantilever arm, which is arranged and/or mounted at an end area on the second connection area. The second connection area can thus form the center of the second holding unit. However, the second connection area does not have to be arranged in the center of gravity of the second holding unit, but can also be arranged outside the center of gravity of the second holding unit.

In addition, the radiation source can be connected to the second holding means, preferably at a further front end area. In particular, the radiation source can have two front end areas, each of which is connected to the first and second holding means.

The connection to the second holding means can be designed in particular on the one hand releasably and on the other hand positively, non-positively and/or frictionally. In this context, it is understood that the further end face of the radiation source does not necessarily have to be connected to the second holding means, but the further front end area ultimately comprises the region of the radiation source that includes the further end face.

Like the first holding means, the second holding means can also have at least one fastening means, for example at least one clip, at least one clamp, at least one spring leg or the like, for fastening to the radiation source, which is/are designed for releasable arrangement of the radiation source. This fastening means can furthermore preferably be arranged displaceably on the second holding means, in particular so that an adjustment of the arrangement of the radiation sources can take place. Alternatively or additionally, it can be provided that the fastening means is formed integrally with the further components of the second holding means. In this context, it is understood that the fastening means are considered to be a component of the second holding means.

In addition, according to the invention, the radiation source can be connected to the second holding means by different types of fastening. Preferably, the fastening means of the second holding means are at least substantially identical in construction to the fastening means of the first holding means.

Furthermore, it can be provided that the radiation source is accommodated in the second holding means at least in certain areas, in particular so that the end face protrudes from the second holding means. The second holding means can also have corresponding fastening means, for example tension clamps, to hold the radiation source.

Furthermore, in another preferred embodiment, it is provided that the second holding means, preferably all second holding means, is connected at one end area to the second connection area. Alternatively or additionally, it may be provided that the second holding means is connected at its free end area—in particular the non-supported end area—to the radiation source. Also in this context, it is understood that the end area of the second holding means does not necessarily have to reference the outermost end of the second holding means, but the end area may comprise the outermost end and an adjoining region. In this case, the end area of the second holding means extends over at most 30%, preferably at most 20%, of the length of the elongated second holding means.

In a further preferred embodiment, it is provided that at least one second holding means, preferably at least two second holding means, preferably all second holding means, can be adjusted via a respective second adjusting means connected to the second connection area. The second adjusting means may, for example, comprise a screw connection, preferably in combination with an elongated hole, and/or a telescopic adjusting means or the like.

Finally, the second adjusting means can be designed in such a way that the oblique position of the central axis of the radiation source attached to the first holding means can be adjusted in relation to the central axis of the reflector. In this context, it is understood that the second adjusting means is “activated” and/or released only as needed. For example, in the released state, the second adjusting means can allow an adjustment of the inclined position of the central axis of the respective radiation source attached to the second holding means, wherein after the alignment has been completed, a renewed locking and/or securing of the second adjusting means can take place—for example, by a tightening of the screw connection. Thus, in the fixed state of the second adjusting means, the radiation source can be firmly arranged on the second holding means, in particular so that no renewed adjustment of the oblique position of the central axis of the radiation source is given.

As explained above, the second holding means can be elongated. Alternatively or additionally, it may be provided that at least two second holding means have a length differing from each other. Alternatively or additionally, it may be provided that the second holding means has at least two second arrangement areas, preferably for arranging the fastening means, for connection to the radiation source.

Preferably, the second holding unit is designed in such a way that at least two angles enclosed between two directly adjacent second holding means are designed differently from one another and, in particular, deviate and/or differ from one another by at least 5%, preferably at least 10%. Particularly preferably, all second holding means are arranged relative to each other in such a way that all included angles between directly adjacent second holding means are different from each other. This can contribute to the further advantageous effect that constructive interference effects can be achieved by the oblique arrangement of the radiation sources in the reflector. This effect can now be further enhanced by the different arrangement of the second holding means, preferably at the second connection area.

Preferably, the first holding unit, in particular the connection area of the first holding unit, is connected to the second holding unit, in particular to the second connection area, via an elongated connecting part. The connecting part can in particular be of rigid and/or stable design. Preferably, the connecting part is reflective on its outer side facing the reflector, preferably with a reflectance of at least 0.6, preferably of at least 0.8.

The connecting part can ensure the stability of the entire radiation unit and/or lamp package. Thus, the connecting part enables the first holding unit and the second holding unit to be connected to each other not only via the radiation sources, but also via the connecting part. The connecting part can in principle be of different shapes, for example cylindrical, tubular or the like.

In particular, the connecting part can be arranged centrally between the radiation sources and/or surrounded by the radiation sources. Thus, the connecting part can be arranged in the central area and/or in the center of the radiation unit and/or the lamp package.

Further in particular, the connecting part is not directly connected to the first and/or second holding means and/or to the respective radiation sources. Preferably, the connecting part is arranged at the connection areas of the first and second holding means. The connecting part thus likewise enables simple assembly and disassembly of the entire lamp package and, in addition, also fixation of the inclined position of the radiation sources, in particular when flow passes through the reflector. In this way, for example, it is possible to prevent the radiation sources from “wobbling” with regard to their orientation in relation to the central axis of the reflector—due to the flow of the medium acting on the radiation source. Thus, the connecting part contributes not only to the stability but also to the improved functioning of the entire irradiation device.

It is particularly preferred that the second holding unit is at least substantially identical in design to the first holding unit. In particular, the second holding unit can be designed complementary and/or mirrored to the first holding unit. Furthermore, the second holding unit can be designed in such a way that the skewing and/or twisting and/or tording of the radiation sources relative to one another can be ensured.

In particular, it is envisaged that no power and/or energy supply is provided to the radiation sources via the second holding unit. The energy and/or power supply of the radiation sources can be ensured in particular via energy supply lines provided in the first holding unit. The second holding unit is ultimately used for fixing and/or securing and/or for stable alignment of the radiation sources in the reflector.

The second holding unit may further be located on another area, such as on the opposite side of the reflector and/or the housing.

Preferably, the first holding unit and/or the first connecting means is connected to a first connection section. The first connection section may be detachably connectable to the housing and/or to the reflector. In particular, the first connection section at least partially protrudes or extends along the outside of the housing. The first connection section thus enables the first holding unit to be connected to the outside, in particular to the outside of the housing facing away from the inside of the reflector.

Furthermore, a first supply device, preferably comprising a plurality of ballasts, in particular for operating the radiation sources, can be arranged and/or connected on the outside of the first connection section. Thus, the first connection section can also serve as a “docking area” for power supply units, ballasts and the like, which are arranged in a supply device, for example. The first supply device can further be electrically connected to the first holding unit via the first connection section.

In addition, the holding system and/or holding device can also be formed in a modular manner by means of various connection sections which, in particular, are arranged directly adjacent to one another and can preferably be connected to one another in a fixed and detachable manner. For example, the second holding unit and/or the second connecting means may be connected to a second connection section. The second connection section may also be detachably connectable to the housing and/or the reflector. Preferably, the second connection section also protrudes from the outside of the housing. The second connection section may be indirectly or directly connected to the first connection section.

In the case of an indirect connection, it can be provided that at least one further connection section is provided which can be releasably connectable and/or connected to the first and/or second connection section in a form-fitting and/or friction-fitting and/or force-fitting manner. This ultimately enables the modular structure of the holding device and, moreover, also a comparatively simple variation of the length of the holding device, which can correlate in particular to the length of the radiation source used.

It is particularly preferred that the first and second connection sections are designed in such a way that they can be releasably connectable and/or connected to one another in a form-fitting and/or friction-fitting and/or force-fitting manner. In such a connection, the first and second connection sections could be directly connected to one another.

Locking means may be provided to connect the connection sections.

In particular, the first, second and/or further connection section projects at least partially into the interior of the reflector and/or adjoins the inside of the reflector. The first, second and/or further connection section can also be set back relative to the inside of the reflector, but faces the inside of the reflector, preferably directly. Preferably, the first, second and/or further connection section have, on the outer surface facing the interior of the reflector, a reflective surface which preferably has a directional or direct reflection with a reflectance of at least 0.6, preferably at least 0.8, more preferably at least 0.9.

For example, the reflector can be designed in such a way that it can be mounted on a profile, in particular an aluminum profile. For example, the reflector can be formed as a sheet, preferably aluminum sheet, wherein the shape of the reflector can be made possible by clamping the reflector to the corresponding profile and/or the connection sections. With such a design of the reflector, the connection sections can provide and/or ensure the stability of the entire irradiation device.

Particularly preferably, between 3 to 25, preferably between 4 to 15, more preferably between 5 to 10, radiation sources, first holding means and/or second holding means are provided.

The number of holding means is designed in particular as a function of the number of radiation sources. In this context, it can be provided that there is a “surplus” of first or second holding means, so that, for example, a radiation source does not have to be arranged at each holding means.

Furthermore, the number of radiation sources can be designed depending on the desired UV radiation dose, the length of the reflector, the volume flow of the medium to be treated and the like. Due to the overall modular design of the entire irradiation device that is made possible, it is also possible to customize the irradiation. For example, an addition of radiation sources can also be made possible as required, for example by arranging further holding means at the respective connection area of the first and/or second holding unit or by using already existing “surplus” holding means for mounting the radiation sources.

Preferably, at least two, preferably at least three, in particular all, radiation sources are of identical design. This achieves an improved radiation pattern, since the radiation sources emit at least essentially the same radiation.

In particular, at least one, preferably all, radiation sources has a diameter D between 1 cm to 20 cm, preferably between 2 cm to 10 cm, more preferably between 4 cm to 6 cm. The aforementioned diameter may be the average and/or the maximum diameter of the radiation source. In particular, it is provided that the diameter of the radiation source is at least substantially constant.

Preferably, at least one, preferably all, radiation sources have a length between 0.2 m to 10 m, preferably between 0.5 m to 5 m, more preferably between 1 m to 2 m.

Furthermore, the inner diameter of the reflector, in particular the inner maximum diameter of the reflector, can be between 100 to 1000 cm, preferably between 200 to 600 cm. In particular, with an inner diameter of 250 cm+/−20%, it is envisaged that five radiation sources are used. The larger the inner diameter of the reflector is designed, the more radiation sources can be used. For example, it can be provided that with an inner diameter of the reflector of 350 cm+/−20%, approximately seven radiation sources are used. For example, with an inner diameter of the reflector of 500 cm+/−20%, ten radiation sources can be used. In this context, it is envisaged that the inner diameter of the reflector is designed to be at least essentially constant—namely over the longitudinal extension of the reflector.

It is particularly preferred if an evaluation device is provided for detecting at least one chemical and/or physical variable. The evaluation device can, for example, be arranged in the first connection section and/or in the first holding unit and/or in the second holding unit and/or in the first and/or second connecting means. In particular, the evaluation device may comprise at least one temperature sensor, UV sensor and/or speed sensor. The evaluation device may serve to improve the operation of the irradiation. For example, the chemical and/or physical variables detected by the evaluation device can be used to control and/or regulate the irradiation, the volume flow of the medium and the like, so that in particular an optimized mode of operation can be provided.

The length of the first holding means and/or the second holding means can be between 0.5·D_{R} to 0.99·D_{R}, preferably between 0.1·D_{R} to 0.5·D_{R}. In this context, D_{R} indicates the inner, in particular maximum, diameter of the reflector. Thus, the first and/or second holding means can be arranged centrally in the reflector, as well as off-center in the reflector. Furthermore, the design of the length of the holding means can also vary in the respective holding unit—that is, in the first and/or second holding unit.

Particularly preferably, at least one, and more preferably at least two, in particular all, radiation sources are designed as LEDs. LEDs have proven to be particularly advantageous for irradiation to kill microorganisms in the medium, especially since they are characterized by a long service life.

Furthermore, it is understood that any intermediate intervals and individual values are included in the above-mentioned intervals and range limits and are to be regarded as disclosed as essential to the invention, even if these intermediate intervals and individual values are not specifically indicated.

Further features, advantages and possible applications of the present invention will be apparent from the following description of examples of embodiments based on the drawing and the drawing itself. In this context, all the features described and/or illustrated constitute the subject-matter of the present invention, either individually or in any combination, irrespective of their summary in the claims or their relation back.

### BRIEF DESCRIPTION OF THE DRAWINGS

FIG. 1 is a schematic perspective view of an irradiation device according to the invention;

FIG. 2 is a schematic front view of the irradiation device shown in FIG. 1;

FIG. 3 is a schematic top view of the irradiation device shown in FIG. 1;

FIG. 4 is a schematic side view of the irradiation device shown in FIG. 1;

FIG. 5 is a schematic perspective view of a further embodiment of the irradiation device according to the invention;

FIG. 6 is a schematic perspective view of a further embodiment of the irradiation device according to the invention;

FIG. 7 is a schematic perspective view of a further embodiment of the irradiation device according to the invention;

FIG. 8 is a schematic perspective view of a housing according to the invention;

FIG. 9 is a schematic perspective view of a further embodiment of an irradiation device according to the invention;

FIG. 10 is a schematic side view of the irradiation device shown in FIG. 9;

FIG. 11 is another schematic side view of the irradiation device shown in FIG. 9;

FIG. 12 is a schematic top view of a first holding unit according to the invention;

FIG. 13 is a schematic representation of a holding device according to the invention;

FIG. 14 is a schematic representation of the arrangement of at least one radiation source in the reflector;

FIG. 15 is a schematic representation of the arrangement of two radiation sources in the reflector;

FIG. 16 is a schematic representation of a further embodiment of a first holding means; and

FIG. 17 is a schematic representation of the alignment of a radiation source according to the invention.

### DETAILED DESCRIPTION

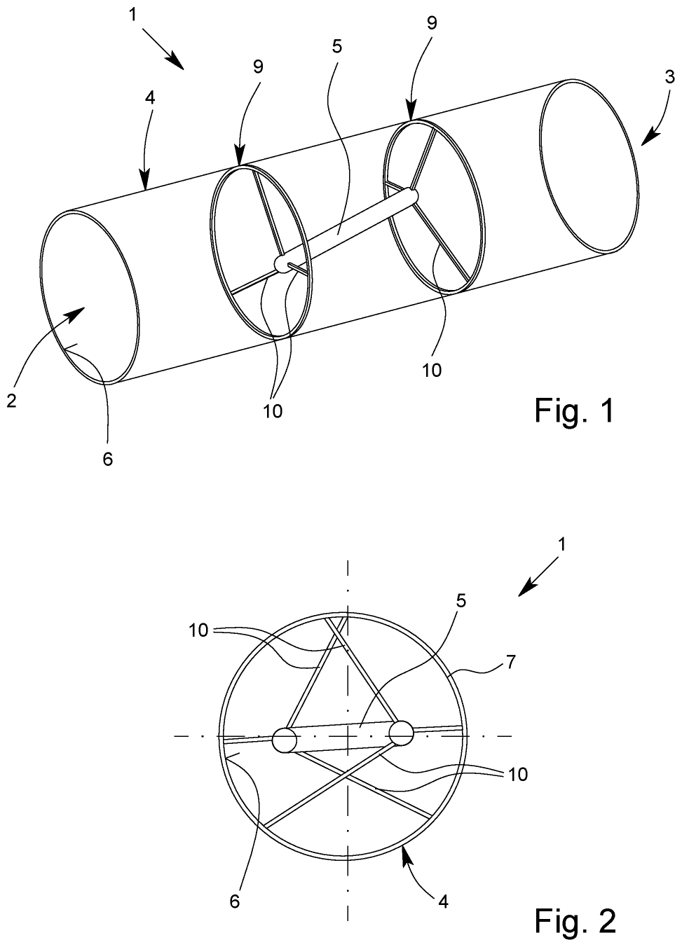

FIG. 1 shows an irradiation device 1 for UV radiation, in particular for UV-C irradiation, of a medium flowing through the irradiation device 1. Water or air can be provided as the medium. In further embodiments, a gas mixture and/or a steam mixture may also be used as medium.

The irradiation device 1 is used in particular to inactivate and/or kill microorganisms present in the medium, such as bacteria, germs, mold and/or viruses.

The irradiation device 1 has an inlet 2 and an outlet 3. The inlet 2 serves for the inlet of the medium and the outlet 3 serves for the outlet of the medium from the irradiation device 1. The irradiation device 1 further comprises a housing 4, which has the inlet 2 and the outlet 3 and through which the medium flows. At least one radiation source 5 is arranged in the housing 4. The radiation source 5 may be connected to and/or attached to and/or mounted on the housing 4. FIG. 1 shows that the two end faces of the radiation source 5 are attached to the housing 4.

On the inner side 6 facing the radiation source 5, the housing 4 is reflective at least in certain areas, preferably over the entire surface, with a reflectance for the UV radiation emitted by the radiation source 5, in particular the UV-C radiation, of at least 0.6. In the embodiment example shown in FIG. 1, it is provided that the reflectance is at least 0.7, preferably at least 0.8.

The radiation source 5 is arranged in the housing 4 in such a way that the radiation emitted by the radiation source 5 is, preferably directed, reflected on the inner side 6 of the housing 4 and that the radiation emitted by the radiation source 5 constructively interferes with the, preferably directed, reflected radiation.

In further embodiments, the radiation source 5 may be arranged in the housing 4 in such a way that the radiation emitted by the radiation source 5 is, preferably directed, reflected at the inner side 6 of the housing 4 and that the radiation emitted by the radiation source 5 has a path difference differing from an integer multiple of half the wavelength and/or of half the wavelength and/or that the radiation emitted by the radiation source 5 interferes at least substantially non-destructively with the, preferably directed, reflected radiation, in particular wherein less than 30%, preferably less than 25% and in particular between 0% to 20%, of the radiation emitted by the radiation source 5 interferes destructively.

The inner side 6 of the housing 4 can also be part of an inner wall 7. The inner wall 7 may be formed as a replaceable component and/or as a component removable from the housing 4 in further embodiments not shown. In the illustrated embodiment example, the inner wall 7 and/or the inner side 6 is a reflector which is designed to reflect the radiation emitted by the radiation source 5.

Thus, an interference image and/or interference pattern characterized by constructive interference can be generated in the interior of the housing 4 and/or in the treatment chamber 8 formed in the interior. In particular, the radiation source 5 is arranged in the housing 4 in such a way that the constructive interference exceeds the destructive interference, so that the advantageous properties of the constructive interference can be used. Preferably, the constructive interference exceeds the destructive interference by at least 10%, preferably by at least 50%.

Finally, the radiation source 5 may be arranged in the housing 4 such that the fraction of interacting and constructively interfering radiation exceeds the fraction of interacting and destructively interfering radiation, preferably by at least 10%, preferably by at least 20%, more preferably by at least 50%.

FIG. 1 shows that the radiation source 5 is arranged in the housing 4 in such a way that the interference pattern produced by interaction of the radiation emitted by the radiation source 5 with the, preferably directed, reflected radiation has a, in particular averaged, maximum total intensity of the interference pattern which is greater by at least 50%, preferably between 300% to 500%, than the maximum intensity of the direct radiation emitted by the radiation source 5 before reflection on the inside of the housing 4.

Furthermore, it is shown in FIG. 1 that the radiation source 5 is attached to the inner side 6 of the housing 4 by a basic holding device 9. In this context, the basic holding device 9 may comprise a plurality of basic holding means 10. In the illustrated embodiment example, the basic holding means 10 are designed as strut-shaped holders. In the embodiment example shown in FIG. 1, it is shown that the front side or the front end area of the radiation source 5 can be fastened to the inner side 6 with at least two, in particular three, basic holding means 10 of the basic holding device 9.

In particular, the radiation source 5 can be fixed to the housing 4 in a rotationally fixed manner via the basic holding device 9. The holding device 9 can further be designed in such a way that the generated radiation pattern and/or interference pattern in the treatment chamber 8 is impaired as little as possible.

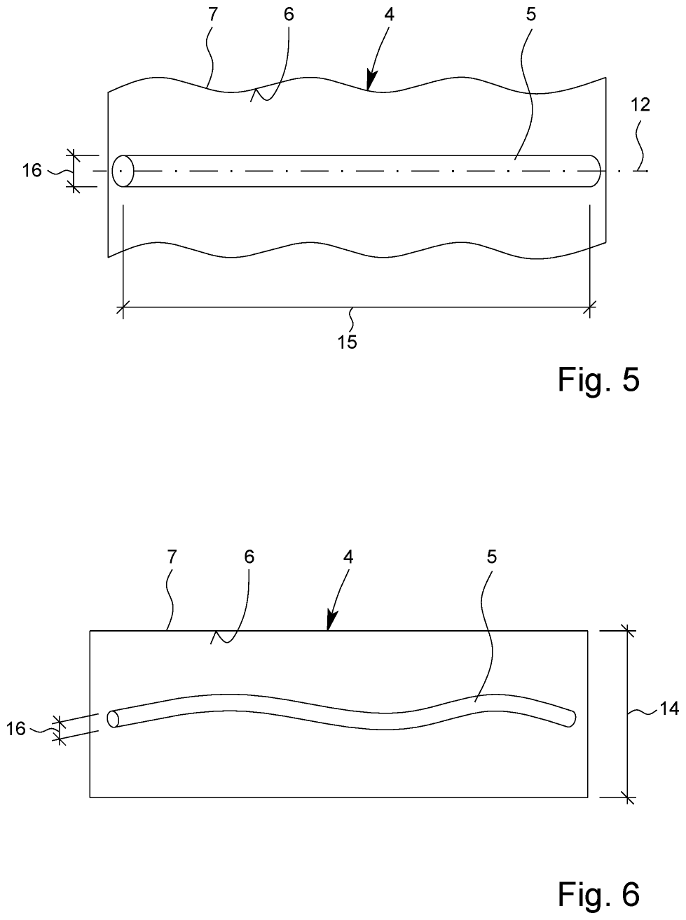

In the embodiment shown in FIG. 5, the inner side 6 has a reflectance of at least 0.7. In further embodiments, the reflectance can be at least 0.8, in particular at least 0.9. The reflectance ultimately indicates the proportion of the radiation emitted by the radiation source 5 that is reflected at the inner side 6.

Although it is not shown, in further embodiments it may be provided that the inner side 6 of the housing 4 and/or the inner wall 7 of the housing 4 is coated with a UV radiation-reflecting coating at least in certain areas, in particular completely. The inner wall 7 can be designed as a removable component or as a reflector.

Furthermore, it is not shown that the inner wall 7 and/or the inner side 6 can have metal as material and/or can consist thereof. In further embodiments, the inner wall 7 may be a sheet metal, in particular an aluminum sheet metal and/or a galvanized sheet metal and/or a stainless steel sheet metal.

FIG. 5 shows that the inner wall 7 of the housing 4 is corrugated. Ultimately, in the embodiment example shown in FIG. 5, the inner wall 7 can have such a shape that the constructive interference in the treatment chamber 8 is increased.

In the embodiment example shown in FIG. 6, the inner wall 7 of the housing 4 and/or also the inner side 6 of the housing 4 is flat—i.e. flat and without elevations.

FIG. 1 shows that the inner wall 7 of the housing 4 is cylindrical in shape.

Finally, in further embodiments, the inner wall 7 and/or the inner side 6 may also have a plurality of elevations and/or depressions.

The inner wall 7 shown in FIG. 1 is also rotationally symmetrical. However, it is understood that the inner wall 7 does not necessarily have to be symmetrical.

From FIGS. 2 to 4 it can be clearly seen that the radiation source 5 is positioned in the housing 4 or in the treatment chamber 8 in such a way that there is no symmetrical arrangement of the radiation source 5 in the housing 4.

In FIG. 4, it is shown that the longitudinal axis 11 of the housing 4 is oriented offset from the longitudinal axis 12 of the radiation source 5. The longitudinal axis 11 of the housing 4 is arranged in the direction of greatest extension. In the illustrated embodiment example, the longitudinal axis 11 also corresponds to the symmetry axis of the housing 4. The longitudinal axis 12 of the radiation source 5 is also arranged in the longitudinal extension of the radiation source 5. In the illustrated embodiment example, the longitudinal axes 11 and 12 are oriented at an angle to each other. They can include an angle α between 3° to 30°, as also illustrated in FIG. 4.

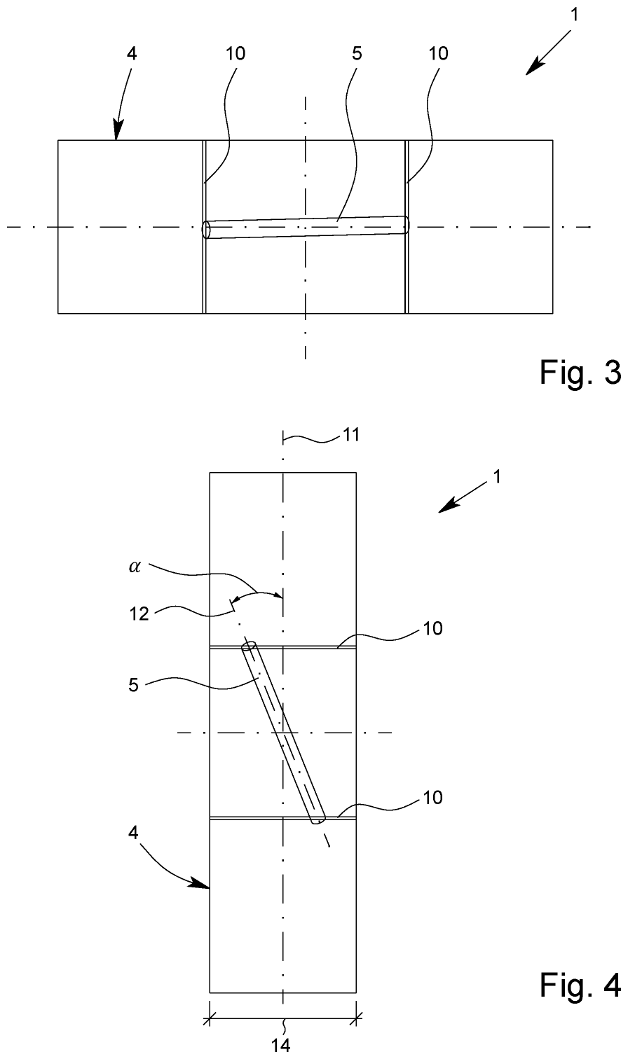

FIG. 3 shows that there is no displacement of the axes of the radiation source 5 and the housing 4 in the top view. In the embodiment example shown in FIGS. 1 to 4, the radiation source 5 is ultimately displaced in a plane relative to the housing 4.

FIG. 1 shows that the radiation source 5 is decentralized with respect to the housing 4.

The basic holding device 9 is designed in such a way that the oblique arrangement of the longitudinal axes 11 and 12 relative to each other can be ensured. In further embodiments, it may be provided that an adjustment or change of the inclined position of the radiation source 5 can be effected via an adjustment of the basic holding device 9. Accordingly, in further embodiments, the basic holding device 9 may be adjustable and/or configured to adjust the radiation source 5.

The inclined position of the radiation source 5 may be selected to achieve the desired interference effects by constructive interference.

Furthermore, FIG. 4 shows that the longitudinal axis 11 of the housing 4 is not coaxial with the longitudinal axis 12 of the radiation source 5. Furthermore, the longitudinal axes 11 and 12 are also not parallel to each other.

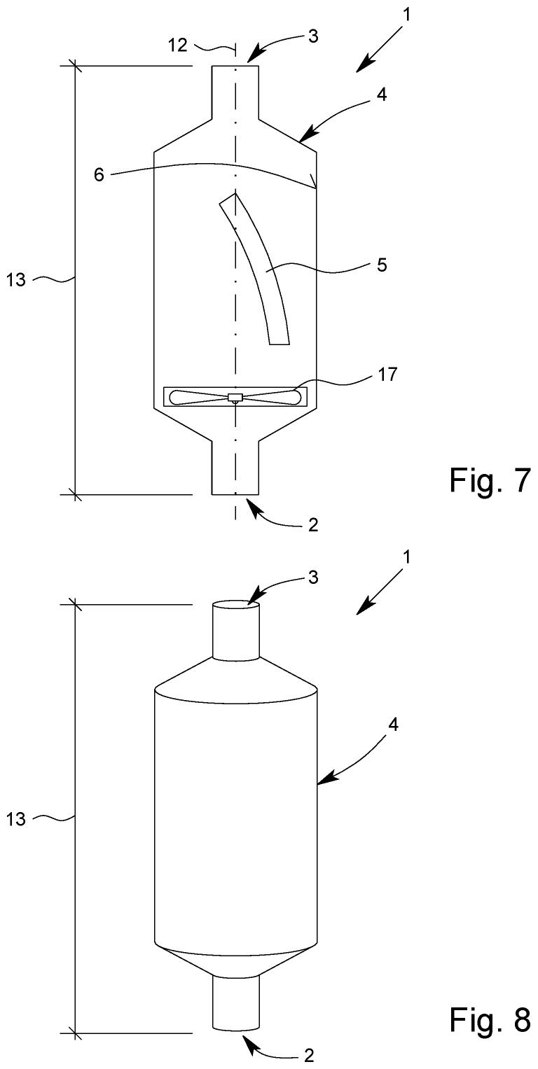

The housing 4 can have a length 13 between 30 to 200 cm. The length 13 is shown schematically in FIGS. 7 and 8. The housing 4 can have a diameter 14, in particular an inner diameter, between 8 and 30 cm.

It is not shown that the radiation source 5 can have a plurality of illuminants, preferably LEDs. In this case, the plurality of illuminants of the radiation source 5 can be arranged in an “array”, in particular directly next to each other, so that in particular an elongated shape of the radiation source 5 results.

In the illustrated embodiment, it is provided that the radiation source 5 has an elongated and rod-like shape.

The length 15 of the radiation source 5 can range from 5 cm to 30 cm.

In further embodiments, the diameter 16 of the radiation source 5 may be between 1 to 20 cm, in particular 5 cm+/−1 cm.

It is also not shown that a plurality of radiation sources 5 are arranged in the housing 4. Consequently, the radiations emitted by the radiation sources 5, in particular the UV-C radiation, can influence each other or interact with each other. The multiple radiation sources 5 can ultimately increase the power of the UV radiation provided. The radiation sources 5 can be arranged (relative) to each other in such a way that the required interference properties can be achieved. Thus, between 2 to 10 radiation sources 5 can be arranged in the treatment chamber 8.

In addition, it is not shown that in the case of a plurality of radiation sources 5, these are arranged overlapping at least in certain areas in the housing 4. The length of the overlapping area may correspond to between 30 and 80% of the length 15 of at least one radiation source 5. The radiation sources 5 can also be arranged at an angle and/or at an angle and/or offset to one another, in particular the longitudinal axes 12 of the radiation sources 5 are arranged offset and/or at an angle to one another. The arrangement of the radiation sources 5 can be done with regard to the interference pattern generated.

FIG. 7 shows that the irradiation device 1 comprises a fan 17 for generating a medium flow from the inlet 2 to the outlet 3. In the illustrated embodiment, the fan 17 is arranged downstream of the inlet 2. In further embodiments, the fan 17 may also be arranged upstream of the inlet 2.

The UV radiation provided by the radiation source 5 can be in a wavelength range of at least 240 nm to 300 nm, in particular in a wavelength range of 250 to 285 nm and in particular at 254 nm+/−10% and/or at 278 nm+/−10%.

Furthermore, it is not shown that a prefilter can be arranged upstream of the inlet 2. The prefilter can be designed in such a way that particles with a diameter greater than 1 μm, preferably greater than 0.5 μm, are filtered out of the medium flow.

The medium flow can have a flow velocity of between 1 and 2 m/s, in particular of 1.7 m/s+/−20%.

In further embodiments, the radiation source 5 may have a power of between 50 to 500 W, preferably between 80 to 200 W. In particular, at least 10%, preferably between 20% to 50%, further preferably at least substantially between 30% to 40%, of the power of the radiation source 5 may be allocated to the portion of the power for the UV-C radiation provided.

The flow of the medium flow in the treatment chamber 8 can be laminar and/or turbulent. Finally, the flow can also be influenced by the fan 17, which ensures the flow through the housing 4.

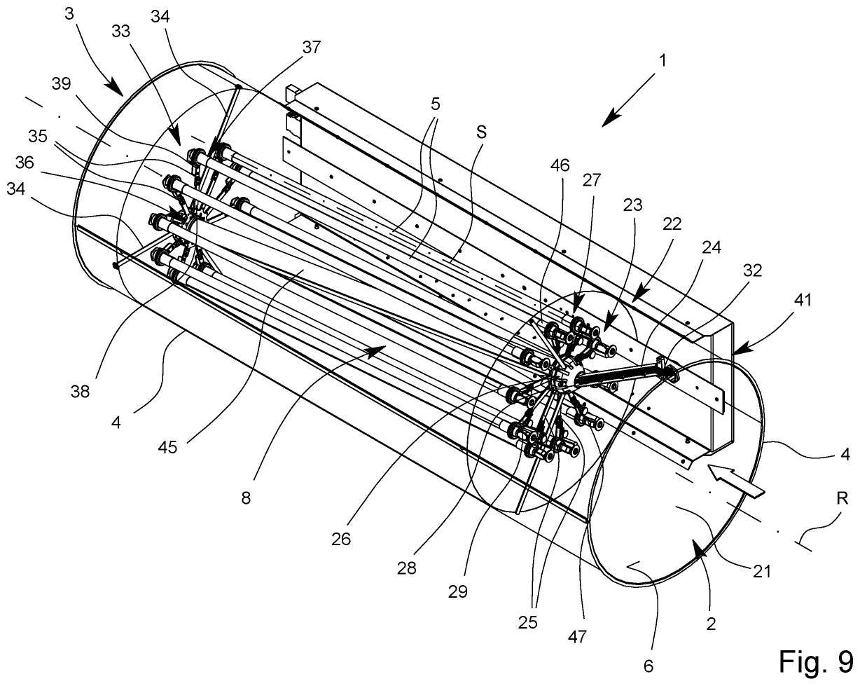

In FIGS. 9 to 16, a further embodiment of an irradiation device 1 is shown, which in particular can be implemented independently of the previously described embodiment of the irradiation device 1 according to FIGS. 1 to 8. In this context, it is understood that explanations as made for the irradiation device 1 according to FIGS. 1 to 8 may—but need not—also apply to the further embodiment of the irradiation device 1 described below. In order to avoid unnecessary repetition, the individual features in this regard will not be discussed again.

FIG. 9 shows an irradiation device 1 which is designed for UV irradiation, in particular UV-C irradiation, of a medium flowing through the irradiation device 1. The medium can be a fluid or a gas. In particular, water or air is provided as the medium. The irradiation device 1 is used for inactivating microorganisms present in the medium, such as bacteria, germs, mold and/or viruses. In particular, the irradiation device 1 is used for inactivating corona viruses. Corona viruses are understood to be, in particular, SARS-CoV-2 viruses.

The irradiation device 1 has a housing 4 which has an inlet 2 and an outlet 3 for the medium. In FIG. 9, the direction of flow of the medium is shown schematically by flow arrows.

At least one radiation source 5 is arranged in the housing 4, namely in the interior of the housing 4. The interior of the housing 4 comprises the treatment chamber 8 in which the radiation source(s) 5 is/are arranged.

The radiation source 5 is used to irradiate the medium flowing through the housing 4.

In the embodiment shown in FIG. 9, a plurality of radiation sources 5 are provided.

The housing 4 has a reflector 21. The inner side 6 of the reflector 21 also forms the inner side 6 of the housing 4. In the illustrated embodiments according to FIGS. 9 to 11, the reflector 21 is shown “transparent” for illustrative reasons.

In particular, the reflector 21 is formed as an aluminum sheet that can be enclosed and/or held in a corresponding profile.

The inner side 6 is designed to be reflective at least in certain areas, preferably over the entire surface, with a reflectance for the UV radiation emitted by the radiation source 5 of greater than 0.6. In particular, the inner side 6 is designed in such a way that a directed, directional reflection of the radiation can take place. For this purpose, the inner side 6 is in particular smooth and flat.

The radiation source 5 is held and/or fixed by a holding device 22. The holding device 22 is connected, preferably detachably, to the housing 4 and/or the reflector 21.

FIG. 9 shows that the holding device 22 is designed in such a way that the central axis S of the at least one radiation source 5 encloses an angle α to the central axis R of the reflector 21.

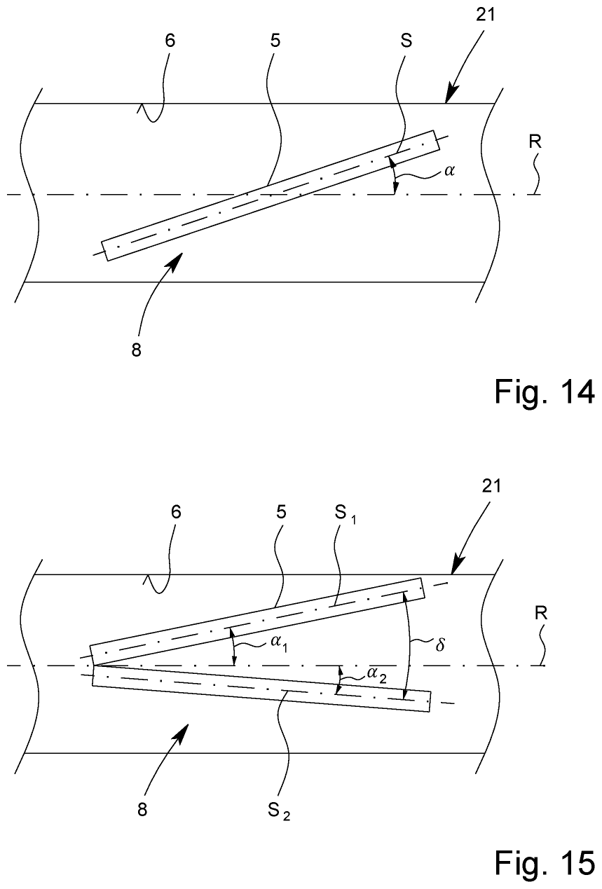

FIG. 14 shows schematically that the radiation source 5 is arranged in such a way that an angle α is enclosed between the central axes S and R. For illustrative reasons, the holding device 22 is not shown in more detail in FIG. 14.

In the embodiment shown in FIG. 14, the included angle α between the central axis S of the at least one radiation source 5 and the central axis R of the reflector 21 is between arcsin((0.2·D/L) and arcsin((4·D)/L). In particular, the angle α is between 2°±0.5°. In order to better represent the inclined position of the radiation source 5 for schematic reasons, the angle α has been deliberately chosen to be larger in the illustrated embodiments according to FIGS. 14 and 15. It is understood, however, that these figures are to be understood as schematic representations and do not reflect the actual proportions.

Furthermore, it is understood that the angle α is particularly of the order of magnitude mentioned above.

Preferably, the angle α is between arcsin(D/L) and arcsin((2·D)/L). Thus, the total skew taken is between D and 2D in particular.

Here D indicates the, in particular maximum and/or average, diameter of the radiation source 5 and L the length of the radiation source 5.

The radiation sources 5 shown in the embodiments illustrated are designed in particular as LED spotlights.

The radiation sources 5 are furthermore rod-shaped and/or cylindrical as well as elongated. The longitudinal extension of the radiation source 5 runs at least essentially in the direction of the longitudinal extension of the reflector 21—taking into account the previously discussed oblique position of the radiation source(s) 5. Thus, preferably no orthogonal arrangement of the radiation source 5 with respect to the central axis R of the reflector 21 is provided.

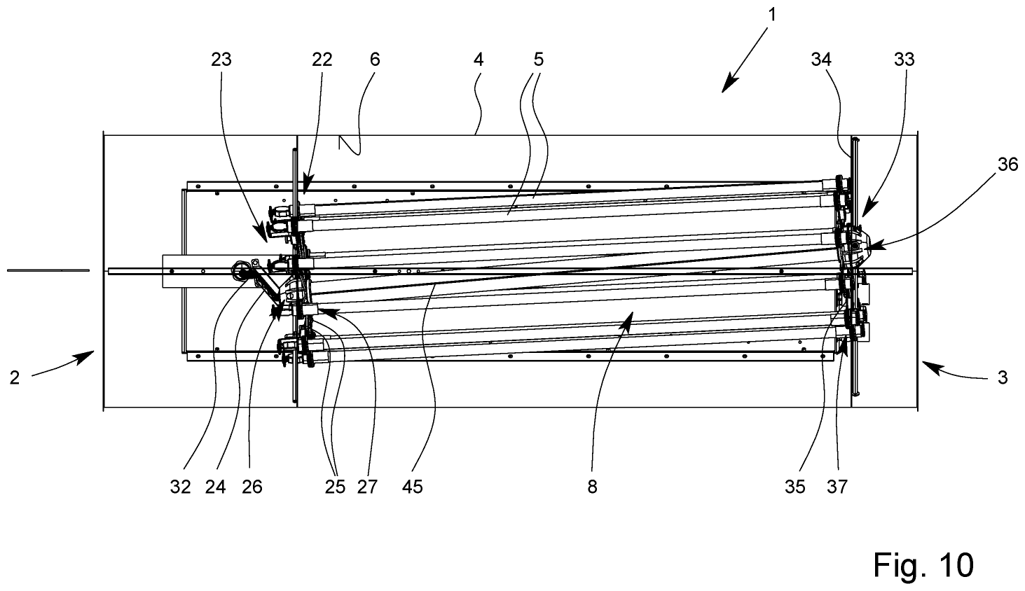

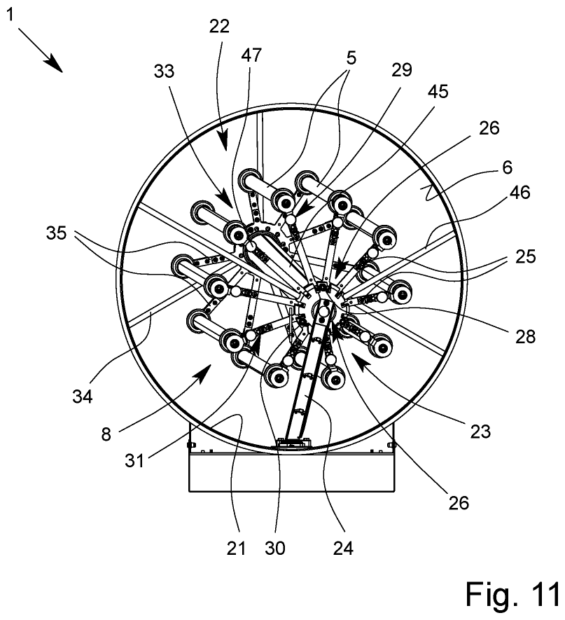

As previously explained, FIGS. 9 to 11 show that a plurality of radiation sources 5 are held and/or fixed to the holding device 22. FIGS. 9 and 10 show corresponding side views of the irradiation device 1 shown in FIG. 9. For example, FIG. 11 shows the inlet 2, with FIG. 10 illustrating the oblique arrangement of the radiation sources 5 by the corresponding side view of the longitudinal side.

In this context, it is understood that in further embodiments a plurality of holding devices 22 can also be provided, wherein at least one radiation source 5, preferably a plurality of radiation sources 5, can be attached to each of the respective holding devices 22. These holding devices 22 can thereby be arranged one below the other and/or next to one another, in particular spaced apart from one another. However, it is particularly preferred that a single holding device 22 is provided.

The radiation sources 5 attached to the holding device 22 can also be referred to collectively as a “lamp package” and/or radiation unit.

The inlet 2 and the outlet 3 may also be located at other positions of the housing 4. Ultimately, the inlet 2 serves to introduce the medium into the treatment chamber 8, while the outlet 3 allows the medium to exit the irradiation device 1. In principle, it can also be provided according to the invention that a plurality of inlets 2 and/or a plurality of outlets 3 are provided.

In the embodiment shown, it is the case that only one radiation source 5 at a time is arranged in the longitudinal direction of the reflector 21 on the holding device 22. The further radiation sources 5 are also aligned at least substantially in the longitudinal direction. It is not shown that in a further embodiment it can also be provided that at least two radiation sources 5 can be arranged one behind the other in the longitudinal direction of the reflector 21 on a holding device 22. Also, a radiation source 5 can be formed in multiple parts.

In the embodiment shown in FIG. 10, each central axis S of each radiation source 5 includes an angle α to the central axis R of the reflector 21. In FIG. 15, it is schematically shown that the central axes S₁ and S₂ respectively include an angle α₁ and α₂ with respect to the central axis R of the reflector 21.

The central axis is understood to be the axis that forms an approximate axis of symmetry of the body. However, non-symmetrical bodies are also considered. In this case, the central axis can run in particular through the center of gravity of the body and in the longitudinal extension of the body. Even deviations from the central axis of ±10% are still subsumed under the “central axis” according to the invention.

FIG. 11 shows schematically that the central axes S of the radiation sources 5 are arranged at least substantially parallel to each other.

FIG. 15 shows schematically that at least two central axes S₁ and S₂ are arranged offset from each other, in particular at an angle. The included angle δ between at least two radiation sources 5 can be between 1° and 50°, in particular between 10° and 40°.

In particular, the central axes S of the radiation sources 5 can also be arranged at an angle and/or skew to each other.

In the case of the holding device 22 shown in FIG. 9, it is provided that this is designed in such a way that the radiation source 5 or the radiation sources 5 are detachably connected to the holding device 22.

The radiation sources 5 can be equally spaced apart. However, it can also be provided that the central axes R enclose a different angle α₁, α₂ to the central axis R of the reflector, as this is shown schematically in FIG. 15, for example. Also, in the embodiment shown in FIG. 15, it is intended that the angles α₁ and α₂—for schematic representation purposes—are deliberately shown “larger” to ultimately clarify the principle.

FIG. 9 shows that the holding device 22 has a first holding unit 23. The first holding unit 23 is detachably connected to the housing 4 and the reflector 21 via a first connecting means 24 of the holding device 22. For further stability of the first holding unit 23, holding webs 46 are also provided, each of which is connected to the housing 4 and/or the reflector 21. The holding webs 46 may be considered to be part of the first connecting means 24.

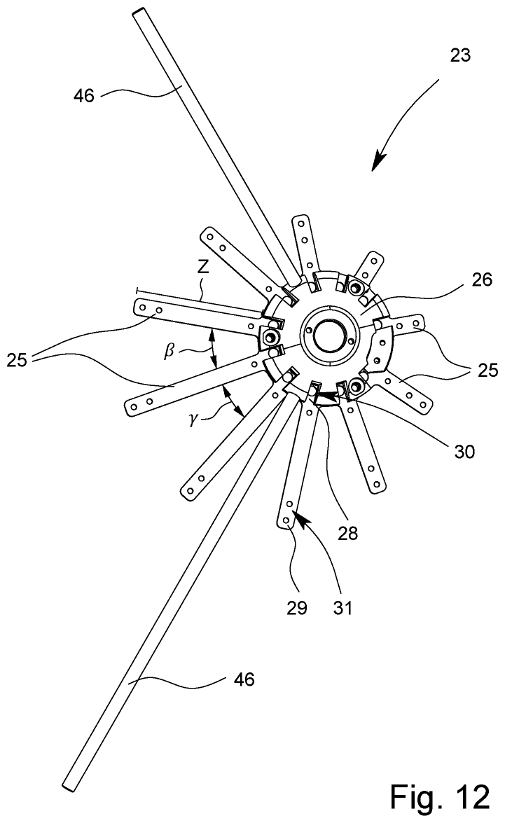

Furthermore, the holding webs 46 are also shown schematically in FIG. 12. FIG. 12 shows the first holding unit 23 without corresponding fastening means 47 for the radiation sources 5.

FIG. 12 shows that the first holding unit 23 has first holding means 25, the first holding means 25 being designed in particular as web-shaped holding arms. The first holding means 25 can be spaced apart from one another at least in certain areas, as can be seen from FIG. 12. The spacing between the first holding means 25 can further vary. Likewise, the included angle β, γ between two immediately adjacent first holding means 25 may vary. In particular, the angles β, γ refer to the central axis of the first holding means 25.

The first holding means 25 can have fastening means 47 for fastening the radiation sources 5. The fastening means 47 are shown schematically in FIG. 9.

The fastening means 47 can be, for example, a clip, a spring leg and/or a tension clamp. Ultimately, different fastening means 47 are possible. The fastening means 47 is in particular a component of the first holding means 25.

In FIG. 11, it is shown that the first holding means 25 are connected to a first connection area 26 of the first holding unit 23. Starting from this connection area 26, the first holding means 25 protrude. One end area 28 of the first holding means 25 is connected to the connection area 26. The first holding means 25 further comprise a further free end area 29, which in turn is provided for arranging the radiation sources 5, in particular the front end areas 27 of the radiation source 5. Thus, the first holding means 25 can in particular be designed as a supporting arm or cantilever arm. The free end area 29 can in particular not be supported or freely arranged. The end area 28 of the first holding means 25 can thereby be arranged directly at the first connection area 26.

In particular, this results in an at least substantially star-shaped and/or sun-shaped configuration of the first holding unit 23, as shown schematically in FIG. 12.

The end area 28 may be supported on or fixedly connected to the connection area 26. It can also be provided that the connection area 26 and the end area 28 are formed integrally with each other.

In the embodiment shown, it is further provided that a first adjusting means 30 is arranged at the end area 28. This first adjusting means 30 enables a relative adjustment to the connection area 26 and, in particular, an adjustment of the radiation source 5 attached to the respective first holding means 25—namely an adjustment of the central axis S of the radiation source 5 with respect to the central axis R of the reflector 21.

It is not shown in more detail that the first holding means 25 are also designed to be telescopic, at least in some areas.

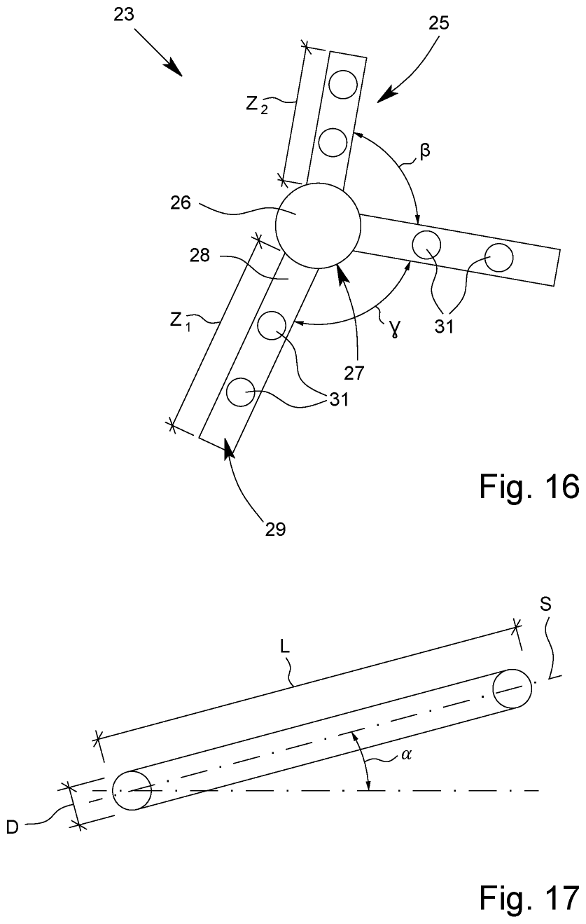

In FIG. 12, it is shown that the first holding means 25 have a different length Z. This is also shown schematically in FIG. 16.

In FIG. 11, it is schematically shown that the radiation source 5 is detachably and frictionally connected to the first holding means 25, in particular to the fastening means 47, at the one front end area 27.

FIG. 16 shows that the first holding means 25 are elongated and that at least two first holding means 25 have a different length Z₁, Z₂. It is further shown schematically in FIG. 16 that a plurality of arrangement areas 31 are provided for each holding means 25. The arrangement areas 31 can be designed for arranging fastening means 47 or for (directly) arranging the front end area 27 of the radiation source 5. For example, the end face of the radiation source 5 can project over the arrangement area 31 and thus also over the holding means 25, in particular if the front end area 27 is accommodated at least in regions in the arrangement area 31 and is held therein, preferably in a frictionally engaged manner. Ultimately, different fastening options are possible between the radiation source 5 and the first holding means 25.

The angles β, γ enclosed between two directly adjacent first holding means 25 can deviate from each other by at least 5% in particular, as shown schematically in FIG. 16.

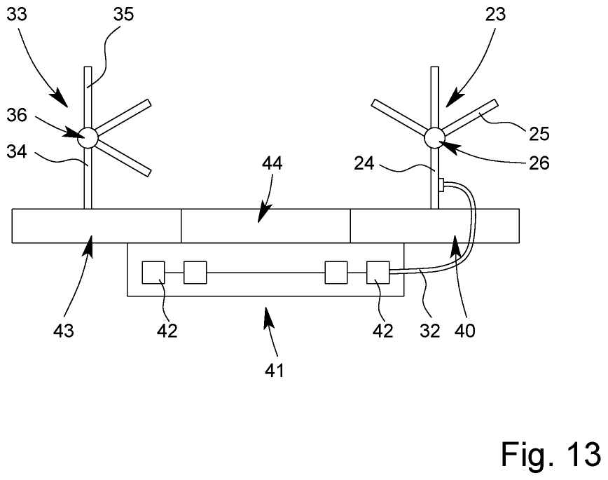

In FIG. 9 it is schematically shown that energy supply lines 32 are provided for supplying energy to the radiation sources 5. These power supply lines 32 are guided in particular along the first connecting means 24 and in particular along the first holding means 25. The energy supply lines 32 can be connected to corresponding power supply units and/or ballasts 42, as can be seen schematically from FIG. 13. In particular, a first supply means 41 is arranged outside the housing 4 on the outer side of the housing 4 facing away from the inner side 6.

Finally, the first holding unit 23 may be designed to supply energy to the radiation sources 5.

FIG. 9 shows that a second holding unit 33 is provided. The second holding unit 33 is detachably connected to the housing 4 and the reflector 21 via a second connecting means 24 of the holding device 22.

According to the embodiment shown in FIG. 9, the second connecting means 24 comprises at least two holding webs which connect the second holding unit 33 to the housing 4 and/or the reflector 21.

FIG. 9 shows that the second holding unit 33 has second holding means 35, the second holding means 35 being designed in particular as web-shaped holding arms. The second holding means 35 can be spaced apart from one another at least in certain areas. The spacing between the second holding means 35 may further vary. Likewise, the included angle between two directly adjacent second holding means 35 can vary.

For fastening the radiation sources 5, the second holding means 35 may have fastening means 47, the fastening means 47 of the second holding means 35 may in particular be designed to correspond to the fastening means 47 of the first holding means 25, so that reference may be made to the preceding explanations.

In FIG. 9, it is shown that the second holding means 35 are connected to a second connection area 36 of the second holding unit 33. Starting from this connection area 36, the second holding means 35 protrude. The second holding means 35 are connected to the connection area 36 by one end area 38. The second holding means 35 further comprise a further free or non-supported end area 39, which in turn is provided for arranging the radiation sources 5, in particular the further front end areas 37 of the radiation source 5.

The end area 38 may be supported on or fixedly connected to the connection area 36. It may also be provided that the connection area 36 and the end area 38 are integrally formed with each other.

It is not shown in more detail that a second adjusting means is arranged at the end area 38. This second adjusting means can be designed in particular in accordance with the first adjusting means 30, so that reference may be made to the explanations on the first adjusting means 30.

It is not shown that the second holding means 35 are also designed to be telescopic, at least in certain areas.

The second holding means 35 can also have a different length Z.

It is not shown in more detail that the second holding means 35 can also have arrangement areas for the radiation source(s) 5. These arrangement areas can be formed like the arrangement areas 31 of the first holding unit 23.

In the embodiment example shown in FIG. 9, the second connecting means 34 is made of several parts and has a plurality of corresponding holding webs. The holding webs of the second connecting means 34 can detachably connect the second holding unit 33 to the housing 4 and/or the reflector 21.

FIG. 9 further shows that the first holding unit 23 is connected to the second holding unit 33 via a connecting part 45. The connecting part 45 can in particular be of elongated design and, in the illustrated embodiment example, connects the first connection area 26 to the second connection area 36. The connecting part 45 is in particular of rigid and stable design. The outer side of the connecting part 45 may be of reflective design.

In FIG. 9, it is shown that the connecting part 45 is arranged in the center of the lamp package and is therefore enclosed and/or surrounded by the radiation sources 5. In particular, the connecting part 45 does not protrude (with respect to the inner side 6) over the radiation sources 5.

It is particularly preferred that the second holding unit 33 is designed to be complementary to the first holding unit 23, in particular so that the desired inclined position of the radiation sources 5 can be achieved.

It is not shown in more detail that the first connecting means 24, the second connecting means 34 and/or the holding webs 46 are designed to be telescopic and/or adjustable. Such an adjustment or telescoping increases in particular the flexibility and/or the adaptability of the entire holding device 22.

FIG. 13 shows a schematic view of the holding means 22 which have not yet been aligned. Finally, the respective radiation sources 5 are not yet arranged to the corresponding holding means 25, 35.

FIG. 13 shows a connection of the radiation sources 5 via power supply lines 32, which are connected to a first power supply device 41, in which several ballasts 42 are arranged. Accordingly, a modular structure of the holding device 22 can be ensured. The modular structure can be adapted in such a way that, in particular, different lengths for the radiation sources 5 can be made possible.

In addition, FIG. 13 shows that the first holding unit 23 and the first connecting means 24 are connected to a first connection section 40. The first connection section 40 is detachably connectable to the housing 4 and/or the reflector 21, which is not shown in more detail. For example, it can be provided that the first connection section 40 has, at least in some areas, a profile for arranging the reflector 21, which can be formed in particular as an aluminum sheet. In principle, however, other embodiments are also conceivable.

In further embodiments, the first connection section 40 protrudes and/or projects at least partially beyond the housing. Thereby, further, the first connection section 40 may have a first supply device 41 on the outside. The first supply device 41 comprises a plurality of ballasts 42, as previously explained. The first supply device 41 is electrically connected to the first connecting means 24 via the power supply lines 32. The power supply lines 32 can be routed through the housing 4, as also shown schematically, for example, in FIG. 9.

Furthermore, FIG. 13 shows that the second holding unit 33 as well as the second connecting means 34 are connected to a second connection section 43. The second connection section 32 may further be detachably connected to the housing 4 and/or the reflector 21 in further embodiments. Furthermore, the second connection section 43 may also protrude over the housing 4 in further embodiments.

The first and second connection sections 40, 43 can be designed in such a way that they can be releasably connectable to one another in a form-fitting and/or friction-fitting and/or force-fitting manner. For this purpose, the connection sections 40, 43 can have corresponding locking contours or the like. FIG. 13 shows that the connection sections 40, 43 can be connected via their end faces. Corresponding locking contours are not shown in more detail in FIG. 13.

FIG. 13 shows that a further connection section 44 is provided for modular assembly. The further connection section 44 can be releasably connectable to the first and/or second connection section 40, 43 in a form-fitting and/or friction-fitting and/or force-fitting manner. For this purpose, the further connection section 44 can have corresponding locking contours which are designed to be complementary to the locking contours of the directly adjacent connection sections.

Not shown in more detail is that the first, second and/or further connection sections 40, 43 and 44 may at least partially extend into the interior of the reflector 21 or be adjacent to—or recessed from—the inner surface 6.

Depending on the embodiment, it may be provided that between 3 to 25 radiation sources 5, first holding means 25 and/or second holding means 35 are provided. The number of radiation sources 5 can depend in particular on the length of the reflector 21, the treated volume flow of the medium and the like. In FIG. 9 it is shown that ten radiation sources 5 are provided.

It is not shown that the number of first holding means 25 and/or second holding means 35 exceeds the number of radiation sources 5. Therefore, it is not mandatory that a radiation source 5 be arranged at each holding means 25. Thus, a “surplus” of holding means 25, 35 can be provided.

In the embodiment shown in FIG. 9, the radiation sources 5 are designed to be identical to each other. In principle, different radiation sources 5 can also be selected if this is desired by the user.

Not shown in more detail is that at least one, preferably all, radiation sources 5 have a diameter D, in particular the maximum and/or the average diameter D, between 1 cm to 20 cm, in particular between 4 cm and 6 cm. Furthermore, the radiation sources 5 may have a length L between 0.2 to 10 m, preferably between 1 to 2 m.

The inner diameter of the reflector 21 can also vary and in particular be between 100 and 1000 cm. In particular, the inner diameter is between 200 to 600 cm.

It is not shown in more detail that an evaluation device for detecting at least one chemical and/or physical variable can be provided. In particular, the evaluation device is arranged in the first connection section 40 and/or in the first holding unit 23. Preferably, the evaluation device comprises a temperature sensor, a UV sensor and/or a speed sensor.

Also not shown in more detail is that the length Z of the first holding means 25 and/or the second holding means 35 is between 0.5·D_{R} to 0.9·D_{R}, preferably between 0.1·D_{R} to 0.5·D_{R}, where D_{R} denotes the inner diameter of the reflector 21, in particular the maximum and/or the average inner diameter of the reflector 21.

### Embodiment Example 1:

In experiments carried out using the irradiation device 1 as shown in FIGS. 1 to 8, it has been shown that with an irradiation device 1 with a power of 190 W+/−10% and an intensity at the surface of the radiation source 5 of about 4233 W/m²+/−10%, the killing rates of the microorganisms indicated below can be achieved as a function of various flow rates of the medium flow. The killing rate of the microorganisms also depends on the resistance of the respective microorganisms to UV-C radiation and on the respective residence time in the housing 4.

The housing 4 has a length of 100 cm with an inner diameter of 15 cm. The radiation source 5 has a diameter of approximately 5 cm+/−10% with a length of 15 cm. The radiation source 5 used has provided UV radiation at a wavelength of 254 nm+/−2%.

Air contaminated with microorganisms has been chosen as the medium.

The table given below presents the experimental results of the achieved killing rate for different microorganisms.

| | | | |
|---|---|---|---|
| Volume flow | Viruses | Bacteria | Fungi |

| | | | | |
|---|---|---|---|---|
| 100 | m³/h | >99.999% | >99.999% | 98.72% |
| 200 | m³/h | >99.999% | >99.999% | 88.68% |
| 300 | m³/h | >99.999% | 99.997% | 76.60% |
| 400 | m³/h | >99.999% | 99.96% | 66.36% |
| 500 | m³/h | >99.999% | 99.82% | 58.17% |
| 600 | m³/h | >99.999% | 99.49% | 51.53% |
| 700 | m³/h | >99.999% | 98.92% | 46.34% |
| 800 | m³/h | 99.999% | 98.10% | 41.99% |
| 900 | m³/h | 99.998% | 97.04% | 38.38% |
| 1,000 | m³/h | 99.995% | 95.79% | 35.32% |

The above table illustrates the particularly efficient virus and bacteria inactivation—even at high volume flows. The aforementioned table further shows that the irradiation device 1 designed according to the invention can efficiently kill not only viruses and bacteria but also fungi.

### Embodiment Example 2:

In experiments conducted with an irradiation device 1 as shown in FIG. 9, it has been shown that with a power of 140 W±10% at an intensity at the surface of the radiation sources 5 of about 4233 W/m²±10%, significantly improved killing rates of microorganisms can be achieved at various flow rates of the medium.

In this case, the angle α between the central axis S of the respective radiation source 5 and the central axis R of the reflector has been between 2° to 10°, in particular 2°±20%.

The housing 4 had a length of 100 cm with an inner diameter of 15 cm. The radiation sources used had a diameter of 5 cm+/−10% with a length of 15 cm. The radiation source 5 used provided UV radiation at a wavelength of 254 nm+/−2%.

In the tests carried out, it was found that an increase in the UV radiation dose can be achieved by the inclined arrangement of the radiation sources 5—in comparison with a “straight” aligned lamp package. By straight alignment it is to be understood that the central axis S of the radiation sources 5 is at least substantially parallel to the central axis R of the reflector.

The irradiation dose could be increased in the conducted experiments from about 600 J/m² with the straight alignment of the lamp package to at least 800 to at least 850 J/m².

The aforementioned UV radiation dose has been determined biodosimetrically—and in particular additionally by a so-called “internal ray tracing” method—and in particular on the basis of the view factor. Finally, the UV radiation dose has been determined in a manner known to the person skilled in the art. With regard to the measurement methods used, reference may be made to DIN(TS) 67506 (in preparation, as of June 2021), which has not yet been completed, and to ISO 15714.

The kill rate of microorganisms was also above 99.99% for viruses at a volumetric flow rate of 700 m³/h. Bacteria could furthermore be killed with a kill rate of 99%. Fungi could further be killed with a kill rate of 50±3%.

Air contaminated with microorganisms has been chosen as the medium.

In the experiments carried out, it was found that due to the oblique arrangement, the constructive interference can advantageously lead to an increase in the UV radiation dose to be administered.

### LIST OF REFERENCE SIGNS

1 Irradiation device
2 Inlet
3 Outlet
4 Housing
5 Radiation source
6 Inner side
7 Inner wall
8 Treatment chamber
9 Basic holding device
10 Basic holding means
11 Longitudinal axis from 4
12 Longitudinal axis from 5
13 Length from 4
14 Diameter from 4
15 Length from 5
16 Diameter from 5
17 Fan
21 Reflector
22 Holding device
23 First holding unit
24 First connecting means
25 First holding means
26 Connection area
27 Front end area of 5
28 End area of 25
29 Free end area of 25
30 First adjusting means
31 Arrangement area
32 Energy supply line(s)
33 Second holding unit
34 Second connecting means
35 Second holding means
36 Second connection area
37 Further front end area of 5
38 End area of 35
39 Free end area of 35
40 First connection section
41 First supply device
42 Ballasts
43 Second connection section
44 Further connection section
45 Connecting part
46 Holding webs
47 Fastening means
α Angle
β Angle between 25
γ Angle between 25
δ Angle between 5
S, S₁, S₂ Central axis of the radiation source
R Central axis of the reflector
D Diameter from 5
L Length from 5
Z, Z₁, Z₂ Length of first and second holding means, respectively

1. An irradiation device for UV irradiation, in particular UV-C irradiation, of a medium flowing through the irradiation device, in particular water or a gaseous medium, preferably air, in particular for inactivating microorganisms present in the medium, such as bacteria, germs, mold and/or viruses, with a housing through which the medium is to flow, having an inlet and an outlet, and at least one radiation source, which is arranged in the interior of the housing and emits UV radiation, for irradiating the medium flowing through the housing;
wherein the housing on the inner side facing the radiation source is designed to be reflective at least in areas, preferably over the entire surface, with a reflectance for the UV radiation emitted by the radiation source of at least 0.6;
wherein the radiation source is arranged in the housing in such a way that the radiation emitted by the radiation source is, preferably directed, reflected on the inner side of the housing and that the radiation emitted by the radiation source constructively interferes with the, preferably directed, reflected radiation; and/or
wherein the radiation source is arranged in the housing in such a way that the radiation emitted by the radiation source is, preferably directed, reflected on the inner side of the housing and that the radiation emitted by the radiation source has a path difference, which differs from the, preferably directed, reflected radiation, of an integer multiple of half the wavelength and/or of half the wavelength and/or that the radiation emitted by the radiation source interferes at least substantially non-destructively with the, preferably directed, reflected radiation, in particular less than 30%, preferably less than 25% and in particular between 0% and 20%, of the radiation emitted by the radiation source interfering destructively.

2. The irradiation device according to claim 1, for UV irradiation, in particular UV-C irradiation, of a medium, in particular fluid or gaseous medium, in particular water or air, flowing through the irradiation device, in particular for inactivating microorganisms located in the medium, such as bacteria, germs, mold and/or viruses, with a housing through which the medium is to flow, having an inlet and an outlet, and at least one radiation source, arranged in the interior of the housing and emitting UV radiation, for irradiating the medium flowing through the housing;
wherein the housing has a reflector, wherein the reflector on the inner side facing the radiation source is designed to be reflective at least in regions, preferably over the entire surface, with reflectance for the UV radiation emitted by the radiation source of at least 0.6;
wherein the at least one radiation source is held and/or fixed by means of a holding device, wherein the holding device is connected, preferably detachably, to the housing and/or the reflector, wherein the holding device is designed in such a way that the central axis (S) of the at least one radiation source encloses an angle (α) to the central axis of the reflector (R).

3. The irradiation device according to claim 2, characterized in that the included angle (α) between the central axis (S) of the at least one radiation source and the central axis (R) of the reflector is between arcsin((0.2·D)/L) and arcsin((4·D)/L), preferably between arcsin((0.5·D)/L) and arcsin((3·D)/L), further preferably between arcsin(D/L) and arcsin((2·D)/L), and/or between 0.5° to 15°, preferably between 2° to 10°, further preferably 2°+/−0.5°, wherein L indicates the length of the radiation source and D indicates the, in particular maximum, diameter of the radiation source.

4. (canceled)

5. The irradiation device according to claim 1, wherein a plurality of radiation sources are held and/or fixed to the holding device, in particular wherein each central axis (S, S₁, S₂) of each radiation source includes an angle (α) to the central axis (R) of the reflector.

6. The irradiation device according to claim 5, wherein the central axes (S, S₁, S₂) of the radiation sources are arranged parallel to one another or in that at least two central axes (S, S₁, S₂) of the radiation sources are arranged offset to one another, preferably obliquely, in particular wherein the included angle (δ) between at least two radiation sources is between 1° and 120°, preferably between 5° and 90°, more preferably between 10° and 40°.

7-9. (canceled)

10. The irradiation device according to claim 2, wherein the holding device has a first holding unit, the first holding unit being releasably connectable to the housing via a first connecting means of the holding device, the first holding unit having a plurality of first holding means spaced apart from one another at least in regions, preferably designed as, in particular web-shaped, holding arms.

11. The irradiation device according to claim 10, wherein the first holding means are connected to a connection area of the first holding unit, in particular wherein the connection area is connected to the connecting means and/or is formed integrally therewith.

12. The irradiation device according to claim 10, wherein the radiation source is connected, preferably at a front end area, to the first holding means, preferably releasably and positively, non-positively and/or frictionally.

13. The irradiation device according to claim 11, wherein the first holding means is connected with an end area to the connection area and/or that the first holding means is connected at its free end area to the radiation source.

14. The irradiation device according to claim 11, wherein at least a first holding means is adjustable via a first adjusting means connected to the connection area, in particular in such a way that the oblique position of the central axis (S) of the radiation source fastened to the first holding means is adjustable with respect to the central axis (R) of the reflector.

15. The irradiation device according to claim 10, wherein the first holding means are elongated and wherein at least two first holding means have a length (Z) differing from one another and/or wherein at least one first holding means has at least two arrangement areas for connection to the radiation source.

16-17. (canceled)

18. The irradiation device according to claim 11, wherein a second holding unit is provided, the second holding unit being connectable, preferably detachably, to the housing via at least one second connecting means of the holding device, the second holding unit having a plurality of second holding means spaced apart from one another at least in regions, preferably designed as, in particular web-shaped, holding arms.

19. The irradiation device according to claim 18, wherein the first holding unit, in particular the connection area, is connected to the second holding unit, in particular to a second connection area, which in particular connects the second holding means to the second connecting means, via an elongated, preferably rigid, connecting part.

20. The irradiation device according to claim 19, wherein the radiation source is connected, preferably at a further front end area, to the second holding means, preferably releasably and positively, non-positively and/or frictionally; and/or
wherein the radiation source and/or the radiation sources is/are mounted on both sides of the first and the second holding unit, in particular in each case in a front end area.

21. (canceled)

22. The irradiation device according to claim 18, wherein the first holding unit and/or the first connecting means is/are connected to a first connection section, in particular wherein the first connection section can be detachably connected to the housing and/or the reflector and/or in particular wherein the first connection section projects at least partially beyond the housing and/or in particular wherein a first supply device can be arranged and/or connected on the outside of the first connection section, preferably comprising a plurality of ballasts, can be arranged and/or connected on the outside of the first connection section, wherein, preferably, the first supply device is electrically connected to the first holding unit via the first connection section.

23. The irradiation device according to claim 22, wherein the second holding unit and/or the second connecting means is/are connected to a second connection section, in particular wherein the second connection section is detachably connectable to the housing and/or the reflector and/or in particular wherein the second connection section protrudes at least partially beyond the housing, in particular wherein the first and the second connection sections are detachably connectable and/or connected to each other in a form-fitting and/or friction-fitting and/or force-fitting manner.

24. (canceled)

25. The irradiation device according to claim 23, wherein at least one further connection section is provided, which is detachably connectable and/or connected to the first and/or second connection section in a form-fitting and/or friction-fitting and/or force-fitting manner.

26. The irradiation device according to claim 25, wherein the first, second and/or further connection sections projects at least partially into the interior of the reflector and/or adjoins the inner side of the reflector.

27-38. (canceled)

39. The irradiation device according to claim 1, wherein a central axis of the housing, in particular of an inner wall of the housing, is oriented offset, preferably obliquely, with respect to the central axis of the radiation source, in particular wherein the central axis of the housing, in particular of the inner wall of the housing, encloses an angle (α) greater than 2°, preferably between 2° and 50°, more preferably between 3° and 15°, to the central axis of the radiation source; and/or
wherein the radiation source is arranged decentrally with respect to the housing.

40-41. (canceled)

42. The irradiation device according to claim 1, wherein the at least one radiation source emits UV radiation in a wavelength range from at least 240 nm to 300 nm, preferably in a wavelength range from 250 nm to 285 nm, further preferably from 270 nm to 280 nm and in particular from 254 nm+/−10% and/or from 278 nm+/−10%.

43. (canceled)
